(19) United States
(12) Patent Application Publication
(10) Pub. No.: US 2022/0400938 A1
(43) Pub. Date: Dec. 22, 2022

(54) MEDICAL OBSERVATION SYSTEM, CONTROL DEVICE, AND CONTROL METHOD

(71) Applicant: Sony Group Corporation, Tokyo (JP)

(72) Inventor: Jun ARAI, Tokyo (JP)

(73) Assignee: Sony Group Corporation, Tokyo (JP)

(21) Appl. No.: 17/620,747

(22) PCT Filed: Jun. 19, 2020
(86) PCT No.: PCT/JP2020/024250

§ 371 Date: Dec. 20, 2021

(30) Foreign Application Priority Data
Jun. 27, 2019 (JP) 2019-120348

(51) Int. Cl. A61B 1/045; A61B 1/00

(52) U.S. Cl. CPC A61B 1/045; A61B 1/00188; A61B 1/000095

(57) ABSTRACT

A medical observation system includes: a plurality of types of sensor units that measure information regarding an internal environment; an acquisition unit (131) that acquires individual sensor values of the plurality of types of sensor units; a comparison unit (132) that compares the individual sensor values of the plurality of types of sensor units acquired by the acquisition unit (131); and a determination unit (134) that determines a sensor unit to be used for observing the internal environment among the plurality of types of sensor units based on a comparison result obtained by the comparison unit (132).

### FIELD

The present disclosure relates to a medical observation system, a control device, and a control method.

### BACKGROUND

In the medical field in recent years, there has been proposed a method of using an articulated (multi-jointed) arm (also referred to as a support arm) with various medical units provided at a distal end of the arm in execution of various operations.

For example, Patent Literature 1 discloses a medical robot arm device capable of performing drive control of a distal end unit and an arm unit having a higher degree of freedom in operability.

### CITATION LIST

### Patent Literature

Patent Literature 1: WO 2015/046081 A

### SUMMARY

### Technical Problem

Meanwhile, in the medical field, an endoscope device is used for internal observation of a human. Unfortunately, however, it is sometimes difficult to grasp the status surrounding the endoscope device only with the image captured by the endoscope device. In addition, when an autonomously/semi-autonomously driven arm is used in the future, it is anticipated that there will be a demand for highly accurate generation of an environment map indicating information (three-dimensional information or the like) of the internal environment of the human.

In view of these circumstances, the present disclosure proposes a medical observation system, a control device, and a control method capable of improving the accuracy of the environment map.

### Solution to Problem

To solve the problem described above, a medical observation system includes: a plurality of types of sensor units that measure information regarding an internal environment; an acquisition unit that acquires individual sensor values of the plurality of types of sensor units; a comparison unit that compares the individual sensor values of the plurality of types of sensor units acquired by the acquisition unit; and a determination unit that determines a sensor unit to be used for observing the internal environment among the plurality of types of sensor units based on a comparison result obtained by the comparison unit.

### BRIEF DESCRIPTION OF DRAWINGS

FIG. 1 is a view illustrating an example of a schematic configuration of an endoscopic surgery system to which the technique according to the present disclosure is applicable.

FIG. 2 is a block diagram illustrating an example of a functional configuration of a camera head and a CCU illustrated in FIG. 1.

FIG. 3 is a schematic view illustrating an appearance of a support arm device according to an embodiment of the present disclosure.

FIG. 4 is a schematic view illustrating a configuration of a forward-oblique viewing endoscope according to the embodiment of the present disclosure.

FIG. 5 is a schematic view illustrating a forward-oblique viewing endoscope and a forward viewing endoscope according to the embodiment of the present disclosure in comparison.

FIG. 6 is a diagram illustrating an example of a configuration of a master-slave device according to the embodiment of the present disclosure.

FIG. 7 is a diagram illustrating an example of a configuration of a medical observation system according to the embodiment of the present disclosure.

FIG. 8A is a cross-sectional view of an endoscope device according to each of embodiments of the present disclosure.

FIG. 8B is a front view of a distal end portion of the endoscope device according to the embodiment of the present disclosure.

FIG. 9 is a table illustrating robustness against reliability deterioration factors in various sensors.

FIG. 10 is a block diagram illustrating an example of a configuration of a sensor unit according to the embodiment of the present disclosure.

FIG. 11 is a block diagram illustrating an example of a configuration of a control device according to the embodiment of the present disclosure.

FIG. 12 is a flowchart illustrating an outline of a process flow of the medical observation system according to the embodiment of the present disclosure.

FIG. 13 is a flowchart illustrating an example of a flow of a first process of the control device according to the embodiment of the present disclosure.

FIG. 14 is a flowchart illustrating an example of a flow of a second process of the control device according to the embodiment of the present disclosure.

FIG. 15 is a flowchart illustrating an example of a flow of a third process of the control device according to the embodiment of the present disclosure.

FIG. 16 is a flowchart illustrating an example of a flow of a fourth process of the control device according to the embodiment of the present disclosure.

FIG. 17 is a diagram illustrating an example of a configuration of a medical observation system according to a modification of the embodiment of the present disclosure.

FIG. 18 is a diagram illustrating an example of a configuration of a surgical robot according to the embodiment of the present disclosure.

FIG. 19 is a flowchart illustrating an example of a process flow of a control device according to a modification of the embodiment of the present disclosure.

FIG. 20 is a hardware configuration diagram illustrating an example of a computer that actualizes functions of an information processing device.

### DESCRIPTION OF EMBODIMENTS

Embodiments of the present disclosure will be described below in detail with reference to the drawings. In each of the following embodiments, the same parts are denoted by the same reference symbols, and a repetitive description thereof will be omitted.

The present disclosure will be described in the following order.

1. Configuration example of endoscope system

2. Configuration example of support arm device

3. Basic configuration of forward-oblique viewing endoscope

4. Medical observation system

4-1. Configuration of medical observation system

4-2. Endoscope device

4-3. Sensor unit

4-4. Control device

5. Processes of medical observation system

5-1. Outline of processes of medical observation system

5-2. First process

5-3. Second process

5-4. Third process

5-5. Fourth process

6. Modification of medical observation system

6-1. Configuration of modification of medical observation system

6-2. Surgical arm system

6-3. Processes of modification of medical observation system

7. Hardware configuration

[1. Configuration Example of Endoscope System]

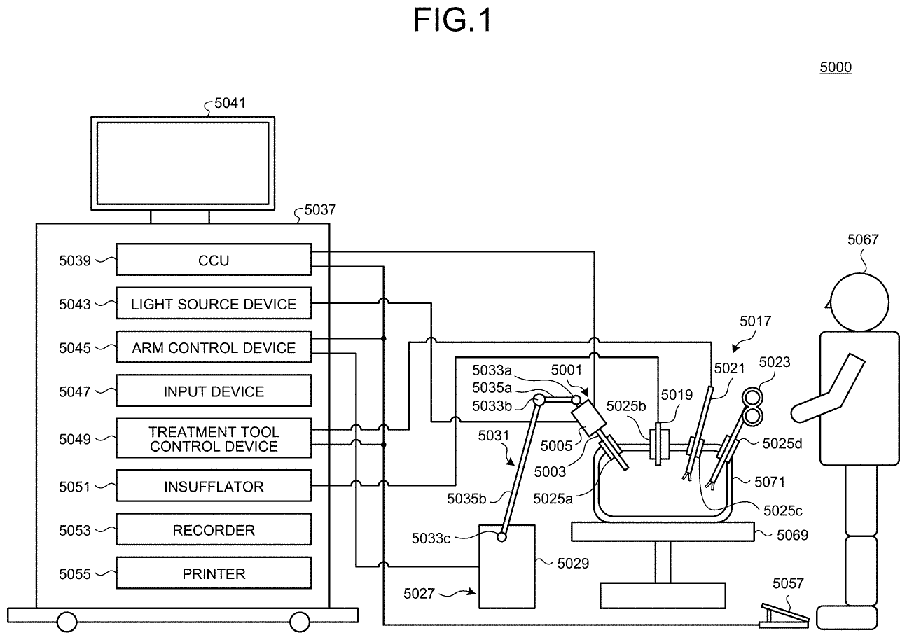

FIG. 1 is a view illustrating an example of a schematic configuration of an endoscopic surgery system 5000 to which the technique according to the present disclosure is applicable. FIG. 1 illustrates a scene in which a surgeon (doctor) 5067 is performing surgery on a patient 5071 on a patient bed 5069 using the endoscopic surgery system 5000. As illustrated, the endoscopic surgery system 5000 includes an endoscope device 5001, other surgical tools 5017, a support arm device 5027 that supports the endoscope device 5001, and a cart 5037 equipped with various devices for endoscopic surgery.

In laparoscopic surgery, a plurality of tubular laparotomy instruments referred to as trocars 5025a to 5025d are punctured into an abdominal wall, instead of performing open surgery of cutting the abdominal wall. Through the trocars 5025a to 5025d, a lens barrel 5003 (that is, an endoscope unit) of the endoscope device 5001 and other surgical tools 5017 are inserted into the body cavity of the patient 5071. In the example of the figure, as other surgical tools 5017, an insufflation tube 5019, an energy treatment tool 5021 and forceps 5023 are being inserted into the body cavity of the patient 5071. Furthermore, the energy treatment tool 5021 is a treatment tool used for incision and detachment of tissues, blood vessel sealing, or the like, by using high-frequency current or ultrasonic vibration. Note that the surgical tool 5017 illustrated in the figure is just an example, and other applicable examples of the surgical tool 5017 include various surgical tools generally used in endoscopic surgery, such as tweezers and a retractor.

An image of the surgical site in the body cavity of the patient 5071 captured by the endoscope device 5001 is displayed on a display device 5041. While viewing the surgical site image displayed on the display device 5041 in real time, the surgeon 5067 performs procedures such as resecting the affected part by using the energy treatment tool 5021 and the forceps 5023. Although not illustrated, the insufflation tube 5019, the energy treatment tool 5021, and the forceps 5023 are supported by the surgeon 5067, assistants, or the like during the surgery.

(Support Arm Device)

The support arm device 5027 includes an arm unit 5031 extending from a base unit 5029. In the illustrated example, the arm unit 5031 includes joints 5033a, 5033b, and 5033c and links 5035a and 5035b, and is driven under the control of an arm control device 5045. The arm unit 5031 supports the endoscope device 5001 and controls its position and posture. This makes it possible to stabilize the position of the endoscope device 5001.

(Endoscope Device)

The endoscope device 5001 includes: a lens barrel 5003 (endoscope unit), a region of a predetermined length from a distal end of which is inserted into the body cavity of the patient 5071; and a camera head 5005 connected to a proximal end of the lens barrel 5003. The example in the figure illustrates the endoscope device 5001 as a rigid endoscope having the lens barrel 5003 of a rigid type. However, the endoscope device 5001 can be a flexible endoscope having a flexible lens barrel 5003.

The distal end of the lens barrel 5003 (endoscope unit) has an aperture to which an objective lens is fitted. The endoscope device 5001 is connected to a light source device 5043. The light generated by the light source device 5043 is guided to the distal end of the lens barrel 5003 by a light guide extending inside the lens barrel 5003, and the guided light will be emitted toward an observation target in the body cavity of the patient 5071 through the objective lens. Note that the lens barrel 5003 connected to the camera head 5005 may be a forward viewing endoscope, a forward-oblique viewing endoscope, or a side-viewing endoscope.

An optical system and an imaging element are provided inside the camera head 5005. Reflected light (observation light) from the observation target is focused on the imaging element by the optical system. The observation light is photoelectrically converted by the imaging element so as to generate an electric signal corresponding to the observation light, that is, an image signal corresponding to the observation image. The image signal is transmitted as RAW data to a camera control unit (CCU) 5039. The camera head 5005 has a function of adjusting a magnification and a focal length by appropriately driving the optical system.

Incidentally, the camera head 5005 may include a plurality of imaging elements in order to support stereoscopic viewing (3D display) or the like. In this case, a plurality of relay optical systems is provided inside the lens barrel 5003 in order to guide the observation light to each of the plurality of imaging elements.

(Various Devices Mounted on Cart)

The CCU 5039 is formed with a central processing unit (CPU), a graphics processing unit (GPU), or the like, and integrally controls operations of the endoscope device 5001 and the display device 5041. Specifically, the CCU 5039 applies, on the image signal received from the camera head 5005, various types of image processing for displaying an image based on the image signal, such as developing processing (demosaicing). The CCU 5039 provides the image signal that has undergone the image processing to the display device 5041. Furthermore, the CCU 5039 transmits a control signal to the camera head 5005 and controls driving thereof. The control signal can include information regarding imaging conditions such as magnification and focal length.

Under the control of the CCU 5039, the display device 5041 displays an image based on the image signal that has undergone the image processing performed by the CCU 5039. When the endoscope device 5001 is a device compatible with high-resolution imaging such as 4K (the number of horizontal pixels 3840×the number of vertical pixels 2160) or 8K (the number of horizontal pixels 7680×the number of vertical pixels 4320), and/or when the endoscope device 5001 is a device compatible with 3D display, for example, the display device 5041 can be a display device capable of high-resolution display and/or capable of 3D display, corresponding to individual specs. When the endoscope device 5001 is a device compatible with high resolution imaging such as 4K or 8K, using the display device 5041 having a size of 55 inches or more can obtain further immersive feeling. Furthermore, the display device 5041 may be provided in plurality, each having different resolutions and sizes for different applications.

The light source device 5043 includes a light source such as Light Emitting Diode (LED), for example, and supplies the irradiation light for imaging the surgical site to the endoscope device 5001.

The arm control device 5045 includes, for example, a processor such as a CPU, and operates according to a predetermined program to control drive of the arm unit 5031 of the support arm device 5027 according to a predetermined control method.

An input device 5047 is an input interface to the endoscopic surgery system 5000. The user can input various types of information and input instructions to the endoscopic surgery system 5000 via the input device 5047. For example, the user inputs various types of information related to the surgery, such as physical information regarding the patient and information regarding the surgical procedure, via the input device 5047. Furthermore, the user inputs, through the input device 5047, an instruction to drive the arm unit 5031, an instruction to change imaging conditions (type of irradiation light, magnification, focal length, or the like) of the endoscope device 5001, and an instruction to drive the energy treatment tool 5021, for example.

The type of the input device 5047 is not limited, and the input device 5047 may be various known input devices. Examples of applicable input devices 5047 include a mouse, a keyboard, a touch panel, a switch, a foot switch 5057, and/or a lever. When a touch panel is used as the input device 5047, the touch panel may be provided on a display surface of the display device 5041.

Alternatively, the input device 5047 is a device worn by the user, such as an eyeglass type wearable device or head mounted display (HMD), for example. Various types of inputs are performed in accordance with user's gesture and line-of-sight detected by these devices. The input device 5047 includes a camera capable of detecting the movement of the user. Various types of inputs are performed in accordance with the user's gesture and line-of-sight detected from a video image captured by the camera. Furthermore, the input device 5047 includes a microphone capable of collecting user's voice, and various inputs are performed by voice through the microphone. In this manner, with a configuration of the input device 5047 capable of inputting various types of information in a non-contact manner, it is possible for the user (for example, the surgeon 5067) located in a clean area to perform non-contact operation of a device located in an unclean area. In addition, since the user can operate the device without releasing a hand from one's surgical tool, leading to enhancement of convenience for the user.

A treatment tool control device 5049 controls the drive of the energy treatment tool 5021 for ablation or dissection of tissue, sealing of blood vessels, or the like. In order to inflate the body cavity of the patient 5071 to ensure a view field for the endoscope device 5001 and to ensure a working space of the surgeon, an insufflator 5051 pumps gas into the body cavity through an insufflation tube 5019. A recorder 5053 is a device capable of recording various types of information associated with surgery. A printer 5055 is a device capable of printing various types of information associated with surgery in various forms such as text, image, graph, or the like.

Hereinafter, a particularly characteristic components of the endoscopic surgery system 5000 will be described in more detail.

(Support Arm Device)

The support arm device 5027 includes the base unit 5029 which is a pedestal, and the arm unit 5031 extending from the base unit 5029. In the illustrated example, the arm unit 5031 is formed with the plurality of joints 5033a, 5033b, and 5033c and the plurality of links 5035a and 5035b coupled via the joints 5033b. However, for the sake of simplicity, FIG. 1 illustrates the configuration of the arm unit 5031 in a simplified manner. In practice, the shapes, the number and the arrangement of the joints 5033a to 5033c and the links 5035a and 5035b, the directions of the rotation axes of the joints 5033a to 5033c, or the like, can be appropriately set so that the arm unit 5031 has a desired degree of freedom. For example, the arm unit 5031 can be suitably configured to have six degrees of freedom, or more. With this configuration, the endoscope device 5001 can be freely moved within the movable range of the arm unit 5031, making it possible to insert the lens barrel 5003 of the endoscope device 5001 into the body cavity of the patient 5071 from a desired direction.

Each of the joints 5033a to 5033c is equipped with an actuator. Each of the joints 5033a to 5033c is rotatable about a predetermined rotation axis by the drive of the actuator. The drive of the actuator is controlled by the arm control device 5045, thereby controlling the rotation angle of each of the joints 5033a to 5033c and controlling the drive of the arm unit 5031. This control can achieve the control of the position and posture of the endoscope device 5001. At this time, the arm control device 5045 can control the drive of the arm unit 5031 by various known control methods such as force control or position control.

For example, the surgeon 5067 may appropriately perform an operation input via the input device 5047 (including the foot switch 5057) so as to appropriately control the drive of the arm unit 5031 by the arm control device 5045 in accordance with the operation input, leading to the control of the position and posture of the endoscope device 5001. With this control, it is possible to move the endoscope device 5001 on the distal end of the arm unit 5031 from a certain position to another certain position, and thereafter fixedly support the endoscope device 5001 at a new position after the movement. Incidentally, the arm unit 5031 may be operated by a method referred to as a master-slave method. In this case, the arm unit 5031 (slave device) can be remotely operated by the user via the input device 5047 (master device) installed at a position in the operating room away from the slave device or at a position away from the operating room.

Furthermore, in a case where the force control is applied, the arm control device 5045 may perform power assist control, in which after receiving an external force from the user, the actuators of the individual joints 5033a to 5033c are driven so as to smoothly move the arm unit 5031 in accordance with the external force. With this control, it is possible to move the arm unit 5031 with a relatively light force when the user moves the arm unit 5031 while directly touching the arm unit 5031. This makes it possible to further intuitively move the endoscope device 5001 with simpler operation, leading to enhancement of convenience for the user.

Here, the endoscope device 5001 is typically supported by a doctor called an endoscopist in endoscopic surgery. In contrast, the use of the support arm device 5027 makes it possible to reliably secure the position of the endoscope device 5001 without manual work, leading to stable acquisition of an image of the surgical site and smooth execution of surgery.

Note that the arm control device 5045 does not necessarily have to be provided in the cart 5037. Furthermore, the arm control device 5045 does not necessarily have to be one device. For example, the arm control device 5045 may be provided in each of the joints 5033a to 5033c of the arm unit 5031 of the support arm device 5027, and the plurality of arm control devices 5045 may cooperate with each other to achieve the drive control of the arm unit 5031.

(Light Source Device)

The light source device 5043 supplies the endoscope device 5001 with irradiation light for imaging the surgical site. The light source device 5043 is formed with, for example, an LED, a laser light source, or a white light source constituted by a combination of these. At this time, in a case where the white light source is constituted with the combination of RGB laser light sources, it is possible to control the output intensity and the output timing of individual colors (individual wavelengths) with high accuracy. Accordingly, it is possible to perform white balance adjustment of the captured image on the light source device 5043. Furthermore, in this case, by emitting the laser light from each of the RGB laser light sources to an observation target on the time-division basis and by controlling the drive of the imaging element of the camera head 5005 in synchronization with the light emission timing, it is also possible to capture the image corresponding to each of RGB colors on the time-division basis. According to this method, a color image can be obtained without providing a color filter on the imaging element.

Furthermore, the drive of the light source device 5043 may be controlled so as to change the intensity of the output light at predetermined time intervals. With the control of the drive of the imaging element of the camera head 5005 in synchronization with the timing of the change of the intensity of the light so as to obtain images on the time-division basis and combine the images, it is possible to generate an image with high dynamic range without so called blackout shadows or blown out highlights (overexposure).

Furthermore, the light source device 5043 may be configured to be able to supply light in a predetermined wavelength band corresponding to special light observation. The special light observation is used to perform narrowband light observation (narrow band imaging). The narrowband light observation uses the wavelength dependency of the light absorption in the body tissue and emits light in a narrower band compared with the irradiation light (that is, white light) at normal observation, thereby imaging a predetermined tissue such as a blood vessel of the mucosal surface layer with high contrast. Alternatively, the special light observation may include fluorescence observation to obtain an image by fluorescence generated by emission of excitation light. Fluorescence observation can be performed to observe fluorescence emitted from a body tissue to which excitation light is applied (autofluorescence observation), and can be performed with local administration of reagent such as indocyanine green (ICG) to the body tissue, and together with this, excitation light corresponding to the fluorescence wavelength of the reagent is emitted to the body tissue to obtain a fluorescent image, or the like. The light source device 5043 can be configured to be able to supply narrow band light and/or excitation light corresponding to such special light observation.

(Camera Head and CCU)

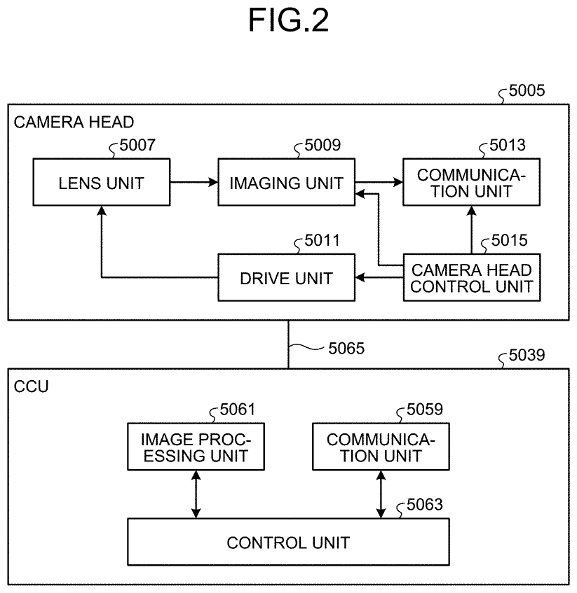

Functions of the camera head 5005 and the CCU 5039 of the endoscope device 5001 will be described in more detail with reference to FIG. 2. FIG. 2 is a block diagram illustrating an example of the configuration of the camera head 5005 and the CCU 5039 illustrated in FIG. 1.

Referring to FIG. 2, the camera head 5005 includes, as functional configuration, a lens unit 5007, an imaging unit 5009, a drive unit 5011, a communication unit 5013, and a camera head control unit 5015. Furthermore, the CCU 5039 includes, as a functional configuration, a communication unit 5059, an image processing unit 5061, and a control unit 5063. The camera head 5005 and the CCU 5039 are connected with each other by a transmission cable 5065 so as to enable bi-directional communication.

First, the functional configuration of the camera head 5005 will be described. The lens unit 5007 is an optical system provided at a connecting portion with the lens barrel 5003. The observation light captured from the distal end of the lens barrel 5003 is guided to the camera head 5005 so as to be incident on the lens unit 5007. The lens unit 5007 is formed by a combination of a plurality of lenses including a zoom lens and a focus lens. The optical characteristics of the lens unit 5007 are adjusted so as to focus the observation light on a light receiving surface of the imaging element of the imaging unit 5009. In addition, the zoom lens and the focus lens are configured to be movable in position on the optical axis in order to adjust the magnification and the focal point of the captured image.

The imaging unit 5009 includes an imaging element and is arranged at a subsequent stage of the lens unit 5007. The observation light having passed through the lens unit 5007 is focused on the light receiving surface of the imaging element, and an image signal corresponding to the observation image is generated by photoelectric conversion. The image signal generated by the imaging unit 5009 is supplied to the communication unit 5013.

An example of the imaging element constituting the imaging unit 5009 is a complementary metal oxide semiconductor (CMOS) image sensor capable of color photography with Bayer arrays. Note that the imaging element may be an imaging element compatible with imaging of a high resolution image of 4K or more. With acquisition of the image of the surgical site with high resolution, the surgeon 5067 can grasp the states of the surgical site in more detail, leading to smooth progress the operation.

In addition, the imaging element constituting the imaging unit 5009 includes a pair of imaging elements for acquiring image signals for the right eye and the left eye corresponding to 3D display. With implementation of 3D display, the surgeon 5067 can grasp the depth of the living tissue in the surgical site with higher accuracy. When the imaging unit 5009 is a multi-plate type, a plurality of lens units 5007 is also provided corresponding to each of the imaging elements.

Furthermore, the imaging unit 5009 does not necessarily have to be provided on the camera head 5005. For example, the imaging unit 5009 may be provided inside the lens barrel 5003 immediately behind the objective lens.

The drive unit 5011 includes an actuator and moves the zoom lens and the focus lens of the lens unit 5007 by a predetermined distance along the optical axis under the control of the camera head control unit 5015. With this operation, the magnification and focal point of the image captured by the imaging unit 5009 can be appropriately adjusted.

The communication unit 5013 includes a communication device for transmitting and receiving various types of information to and from the CCU 5039. The communication unit 5013 transmits the image signal obtained from the imaging unit 5009 as RAW data to the CCU 5039 via the transmission cable 5065. At this time, in order to display the captured image of the surgical site with low latency, the image signal is preferably transmitted by optical communication. This is because, at the time of surgery the surgeon 5067 performs surgery while observing the condition of the affected part using captured images, and thus displaying moving images of the surgical site in real time as much as possible is demanded for safer and more reliable surgery. In a case where optical communication is performed, the communication unit 5013 is provided with a photoelectric conversion module that converts an electric signal into an optical signal. The image signal is converted into an optical signal by the photoelectric conversion module and then transmitted to the CCU 5039 via the transmission cable 5065.

Furthermore, the communication unit 5013 receives a control signal for controlling drive of the camera head 5005 from the CCU 5039. The control signal includes information associated with imaging conditions, such as information designating a frame rate of a captured image, information designating an exposure value at the time of imaging, and/or information designating the magnification and focal point of the captured image. The communication unit 5013 supplies the received control signal to the camera head control unit 5015. Note that the control signal from the CCU 5039 may also be transmitted by optical communication. In this case, the communication unit 5013 is provided with a photoelectric conversion module that converts an optical signal into an electric signal, and the control signal is converted into an electric signal by the photoelectric conversion module and then supplied to the camera head control unit 5015.

Note that the imaging conditions such as the frame rate, the exposure value, the magnification, and the focus are automatically set by the control unit 5063 of the CCU 5039 based on the acquired image signal. That is, an Auto Exposure (AE) function, an Auto Focus (AF) function, and an Auto White Balance (AWB) function are to be installed in the endoscope device 5001.

The camera head control unit 5015 controls the drive of the camera head 5005 based on the control signal from the CCU 5039 received via the communication unit 5013. For example, the camera head control unit 5015 controls drive of the imaging element of the imaging unit 5009 based on information designating the frame rate of the captured image and/or information designating exposure at the time of imaging. Furthermore, for example, the camera head control unit 5015 appropriately moves the zoom lens and the focus lens of the lens unit 5007 via the drive unit 5011 based on the information designating the magnification and the focal point of the captured image. The camera head control unit 5015 may further include a function of storing information for identifying the lens barrel 5003 and the camera head 5005.

Note that arranging the lens unit 5007, the imaging unit 5009, or the like, in a hermetically sealed structure having high airtightness and waterproofness would make it possible to allow the camera head 5005 to have resistance to autoclave sterilization processing.

Next, a functional configuration of the CCU 5039 will be described. The communication unit 5059 includes a communication device for transmitting and receiving various types of information to and from the camera head 5005. The communication unit 5059 receives an image signal transmitted from the camera head 5005 via the transmission cable 5065. At this time, as described above, the image signal can be suitably transmitted by optical communication. In this case, for optical communication, the communication unit 5059 is provided with a photoelectric conversion module that converts an optical signal into an electric signal. The communication unit 5059 supplies the image signal converted into the electric signal to the image processing unit 5061.

Furthermore, the communication unit 5059 transmits a control signal for controlling the drive of the camera head 5005 to the camera head 5005. The control signal may also be transmitted by optical communication.

The image processing unit 5061 performs various types of image processing on the image signal in RAW data transmitted from the camera head 5005. Examples of the image processing include various known signal processing such as development processing, high image quality processing (band enhancement processing, super-resolution processing, noise reduction (NR) processing, camera shake correction processing, and/or the like), and/or enlargement processing (electronic zoom processing). Furthermore, the image processing unit 5061 performs demodulation processing on the image signals for performing AE, AF, and AWB.

The image processing unit 5061 includes a processor such as a CPU and a GPU. The processor operates in accordance with a predetermined program to enable execution of the above-described image processing and demodulation processing. Note that, in a case where the image processing unit 5061 includes a plurality of GPUs, the image processing unit 5061 appropriately divides the information related to image signals, and performs image processing in parallel by the plurality of GPUs.

The control unit 5063 performs various types of control related to imaging of the surgical site by the endoscope device 5001 and display of the captured image. For example, the control unit 5063 generates a control signal for controlling the drive of the camera head 5005. At this time, in a case where the imaging condition has been input by the user, the control unit 5063 generates the control signal based on the input by the user. Alternatively, in a case where the endoscope device 5001 includes the AE function, the AF function, and the AWB function, the control unit 5063 appropriately calculates the optimum exposure value, a focal length, and white balance in accordance with a result of demodulation processing performed by the image processing unit 5061, and generates a control signal.

Furthermore, the control unit 5063 controls the display device 5041 to display the image of the surgical site based on the image signal that has undergone image processing performed by the image processing unit 5061. At this time, the control unit 5063 recognizes various objects in the image of the surgical site by using various image recognition techniques. For example, the control unit 5063 detects the shape, color, or the like of the edge of an object included in the surgical site image, making it possible to recognize a surgical tool such as forceps, a specific living body site, bleeding, occurrence of mist at the time of using the energy treatment tool 5021, or the like. When displaying the image of the operation site on the display device 5041, the control unit 5063 superimposes and displays various surgical operation assistance information on the image of the surgical site by using the recognition result. Surgical assistance information is superimposed and displayed, and presented to the surgeon 5067, thereby making it possible to proceed with surgery more safely and reliably.

The transmission cable 5065 connecting the camera head 5005 and the CCU 5039 is an electric signal cable compatible with electric signal communication, an optical fiber compatible with optical communication, or a composite cable of these.

Here, while the example illustrated in the drawing is a case of performing wired communication using the transmission cable 5065, communication between the camera head 5005 and the CCU 5039 may be performed wirelessly. In a case where the communication between the two units is performed wirelessly, there is no need to dispose the transmission cable 5065 in the operating room, making it possible to eliminate a situation in which the movement of the medical workers in the operating room is hindered by the transmission cable 5065.

An example of the endoscopic surgery system 5000 to which the technique according to the present disclosure can be applied has been described above. Although the endoscopic surgery system 5000 has been described here as an example, the system to which the technique according to the present disclosure can be applied is not limited to such an example. For example, the technique according to the present disclosure may be applied to a flexible endoscope system for examination or a microscopic surgery system.

[2. Configuration Example of Support Arm Device]

Next, an example of a configuration of a support arm device to which the technique according to the present disclosure can be applied will be described below. The support arm device described below is an example configured as a support arm device that supports the endoscope at the distal end of the arm unit. However, the present embodiment is not limited to such an example. Furthermore, in a case where the support arm device according to an embodiment of the present disclosure is applied to the medical field, the support arm device can function as a medical support arm device.

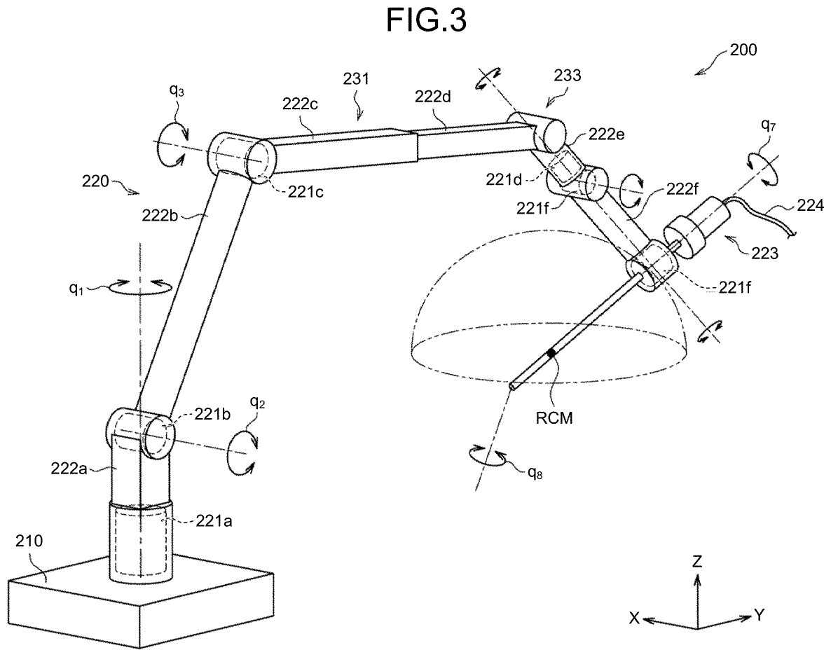

FIG. 3 is a schematic view illustrating an appearance of a support arm device 200 according to the present embodiment. As illustrated in FIG. 3, the support arm device 200 according to the present embodiment includes a base unit 210 and an arm unit 220. The base unit 210 is a base of the support arm device 200, and the arm unit 220 extends from the base unit 210. Furthermore, although not illustrated in FIG. 3, a control unit that integrally controls the support arm device 200 may be provided in the base unit 210, and the drive of the arm unit 220 may be controlled by the control unit. The control unit includes various signal processing circuits such as a CPU and a DSP, for example.

The arm unit 220 includes a plurality of active joints 221a to 221f, a plurality of links 222a to 222f, and an endoscope device 223 as a distal end unit provided at the distal end of the arm unit 220.

The links 222a to 222f are substantially rod-shaped members. One end of the link 222a is coupled to the base unit 210 via the active joint 221a, the other end of the link 222a is coupled to one end of the link 222b via the active joint 221b, and the other end of the link 222b is coupled to one end of the link 222c via the active joint 221c. The other end of the link 222c is coupled to the link 222d via a passive slide mechanism 231, and the other end of the link 222d is coupled to one end of the link 222e via a passive joint 233. The other end of the link 222e is coupled to one end of the link 222f via the active joints 221d and 221e. The endoscope device 223 is coupled to the distal end of the arm unit 220, that is, the other end of the link 222f via the active joint 221f. In this manner, the ends of the plurality of links 222a to 222f are coupled to each other by the active joints 221a to 221f, the passive slide mechanism 231, and the passive joints 233 with the base unit 210 as a fulcrum, thereby forming an arm shape extending from the base unit 210.

The drive control of the actuators provided in the individual active joints 221a to 221f in the arm unit 220 is performed, thereby controlling the position and posture of the endoscope device 223. In the present embodiment, the distal end of the endoscope device 223 enters the body cavity of the patient, which is the operation site, and captures a partial region of the operation site. The distal end unit provided at the distal end of the arm unit 220 is not limited to the endoscope device 223, and an exoscope or a microscope can be used instead of the endoscope. Furthermore, various medical instruments may be connected to the distal end of the arm unit 220 as a distal end unit. In this manner, the support arm device 200 according to the present embodiment is configured as a medical support arm device including a medical instrument.

Hereinafter, the support arm device 200 will be described by defining coordinate axes as illustrated in FIG. 3. Furthermore, the up-down direction, the front-rear direction, and the left-right direction are defined in accordance with the coordinate axes. That is, the up-down direction with respect to the base unit 210 installed on the floor surface is defined as the z-axis direction and the up-down direction. Furthermore, a direction orthogonal to the z-axis and in which the arm unit 220 extends from the base unit 210 (that is, the direction in which the endoscope device 223 is located with respect to the base unit 210) is defined as a y-axis direction and a front-rear direction. Furthermore, a direction orthogonal to the y-axis and the z-axis is defined as an x-axis direction and a left-right direction.

The active joints 221a to 221f pivotably couple the links to each other. The active joints 221a to 221f have actuators, and have a rotation mechanism that is rotationally driven about a predetermined rotation axis by drive of the actuators. By controlling the rotational drive of each of the active joints 221a to 221f, it is possible to control the drive of the arm unit 220, such as extending or contracting (folding) of the arm unit 220, for example. Here, the drive of the active joints 221a to 221f can be controlled by known whole-body cooperative control and idealized joint control, for example. Since the active joints 221a to 221f have the rotation mechanism as described above, the drive control of the active joints 221a to 221f in the following description specifically means the control of the rotation angles and/or generated torques in the active joints 221a to 221f (torques generated by the active joints 221a to 221f).

The passive slide mechanism 231 is an aspect of a passive mode change mechanism, and couples the link 222c and the link 222d so as to be advanceable/retreatable in a predetermined direction. For example, the passive slide mechanism 231 may couple the link 222c and the link 222d to each other so as to be linearly movable. However, the forward and backward movement of the link 222c and the link 222d is not limited to the linear movement, and may be advance/retreat movement in a direction forming an arc shape. The passive slide mechanism 231 is operated to advance/retreat by a user, for example, and makes a distance between the link 222c on one end side of the active joint 221c and the passive joint 233 variable. This makes it possible to change the overall mode of the arm unit 220.

The passive joint 233 is an aspect of the passive mode change mechanism, and pivotably couple the link 222d and the link 222e to each other. Having received a pivot operation from the user, the passive joint 233 makes the angle formed by the link 222d and the link 222e variable. This makes it possible to change the overall mode of the arm unit 220.

Note that, in the present specification, the “posture of the arm unit” indicates a state of the arm unit in which at least a part of the portion constituting the arm can be changed by drive control or the like. As a specific example, the state of the arm unit that can be changed by the drive control of the actuators provided in the active joints 221a to 221f by the control unit in a state where the distance between the active joints adjacent to each other across one or a plurality of links is constant can correspond to the “posture of the arm unit”. In the present disclosure, the “posture of the arm unit” is not limited to the state of the arm unit that can be changed by the drive control of the actuator. For example, the “posture of the arm unit” may be a state of the arm unit, which has been changed by cooperative operation of the passive joints. Furthermore, in the present disclosure, the arm unit does not necessarily have to include a joint. In this case, the “posture of the arm unit” represents a position with respect to a target object or a relative angle with respect to the target object. Furthermore, the “mode of the arm unit” indicates a state of the arm unit that can change together with a change in the relationship between the position and posture of individual parts constituting the arm. As a specific example, the state of the arm unit that can change together with the change in the distance between the active joints adjacent to each other across the link(s) or the angle formed by the links joining the active joints adjacent to each other, along with the operation of the passive mode change mechanism, can correspond to the “form of the arm unit”. Note that, in the present disclosure, the “mode of the arm unit” is not limited to the state of the arm unit that can change together with the change in the distance between the active joints adjacent to each other across the link or the angle formed by the links joining the active joints adjacent to each other. For example, the “mode of the arm unit” may be a state of the arm unit that can change together with the change in a positional relationship or angles between the passive indirect portions by cooperative operations of the passive joints. Furthermore, when the arm unit does not include joints, the “posture of the arm unit” may be a state of the arm unit that can change together with the change in the position with respect to the target object or the relative angle with respect to the target object.

The support arm device 200 according to the present embodiment includes six active joints, namely, the active joints 221a to 221f, achieving six degrees of freedom regarding the drive of the arm unit 220. That is, while the drive control of the support arm device 200 is actualized by the drive control of the six active joints 221a to 221f by the control unit, the passive slide mechanism 231 and the passive joint 233 are not defined as the target of the drive control by the control unit.

Specifically, as illustrated in FIG. 3, the active joints 221a, 221d, and 221f are arranged such that the longitudinal direction of each of the connected links 222a and 222e and the imaging direction of the connected endoscope device 223 are aligned with the rotation axis direction. The active joints 221b, 221c, and 221e are arranged such that the x-axis direction, which is a direction in which the coupling angle of each of the connected links 222a to 222c, 222e, and 222f and the endoscope device 223 is changed in a y-z plane (plane defined by the y-axis and the z-axis), is aligned with the rotation axis direction. In this manner, in the present embodiment, the active joints 221a, 221d, and 221f have a function of performing a motion referred to as yawing, and the active joints 221b, 221c, and 221e have a function of performing a motion referred to as pitching.

With such a configuration of the arm unit 220, the support arm device 200 according to the present embodiment can achieve six degrees of freedom in the drive of the arm unit 220, making it possible to freely move the endoscope device 223 within a movable range of the arm unit 220. FIG. 3 illustrates a hemisphere as an example of a movable range of the endoscope device 223. Assuming that the remote center of motion (RCM) in the hemisphere is an imaging center of the operation site to be imaged by the endoscope device 223, the operation site can be imaged from various angles by moving the endoscope device 223 on the spherical surface of the hemisphere in a state where the imaging center of the endoscope device 223 is fixed to the center point of the hemisphere.

An example of the configuration of the support arm device to which the technique according to the present disclosure can be applied has been described above.

Although the arm unit 220 of the support arm device 200 has been described as having a plurality of joints and having six degrees of freedom, the present disclosure is not limited to these. Specifically, the arm unit 220 is only required to have a structure in which the endoscope device 223 or an exoscope can be disposed at the distal end. For example, the arm unit 220 may have a configuration having only one degree of freedom to allow the endoscope device 223 to drive so as to move forward in a direction of entering the body cavity of the patient and a direction of moving backward.

[3. Basic Configuration of Forward-Oblique Viewing Endoscope]

Next, a basic configuration of a forward-oblique viewing endoscope will be described as an example of the endoscope.

FIG. 4 is a schematic view illustrating a configuration of a forward-oblique viewing endoscope 4100 according to an embodiment of the present disclosure. As illustrated in FIG. 4, the forward-oblique viewing endoscope 4100 is attached to the distal end of a camera head 4200. The forward-oblique viewing endoscope 4100 corresponds to the lens barrel 5003 described with reference to FIGS. 1 and 2, and the camera head 4200 corresponds to the camera head 5005 described with reference to FIGS. 1 and 2. The forward-oblique viewing endoscope 4100 and the camera head 4200 may be configured to be pivotable independently of each other. An actuator may be provided between the forward-oblique viewing endoscope 4100 and the camera head 4200 similarly to the joints 5033a, 5033b, and 5033c. This would allow the forward-oblique viewing endoscope 4100 to rotate with respect to the camera head 4200 by drive of the actuator. With this configuration, a rotation angle θ_{z} to be described below is controlled.

The forward-oblique viewing endoscope 4100 is supported by the support arm device 5027. The support arm device 5027 has a function of holding the forward-oblique viewing endoscope 4100 instead of the endoscopist and moving the forward-oblique viewing endoscope 4100 so as to be able to view a desired site by the operation of the surgeon or the assistant.

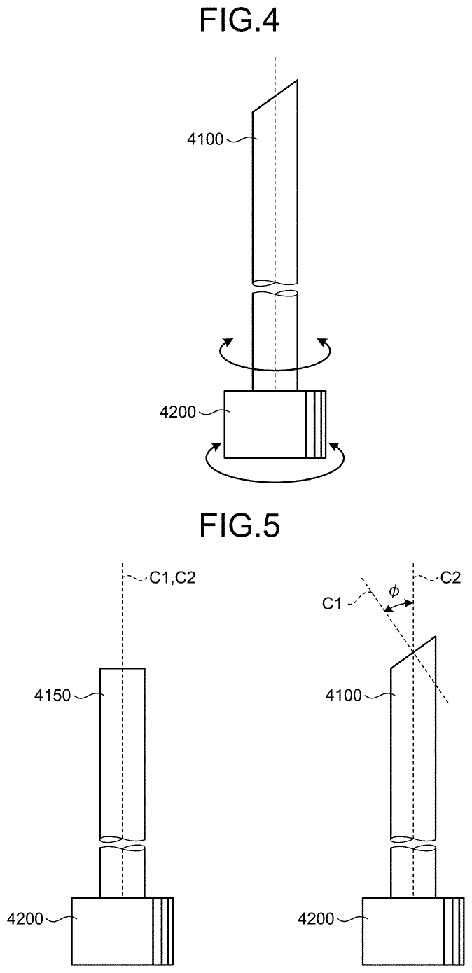

FIG. 5 is a schematic view illustrating the forward-oblique viewing endoscope 4100 and a forward viewing endoscope 4150 in comparison. In the forward viewing endoscope 4150, a direction (C1) of the objective lens toward the subject is aligned with a longitudinal direction (C2) of the forward viewing endoscope 4150. In contrast, in the forward-oblique viewing endoscope 4100, the direction (C1) of the objective lens toward the subject has a predetermined angle ϕ with respect to the longitudinal direction (C2) of the forward-oblique viewing endoscope 4100.

The basic configuration of the forward-oblique viewing endoscope has been described above as an example of the endoscope.

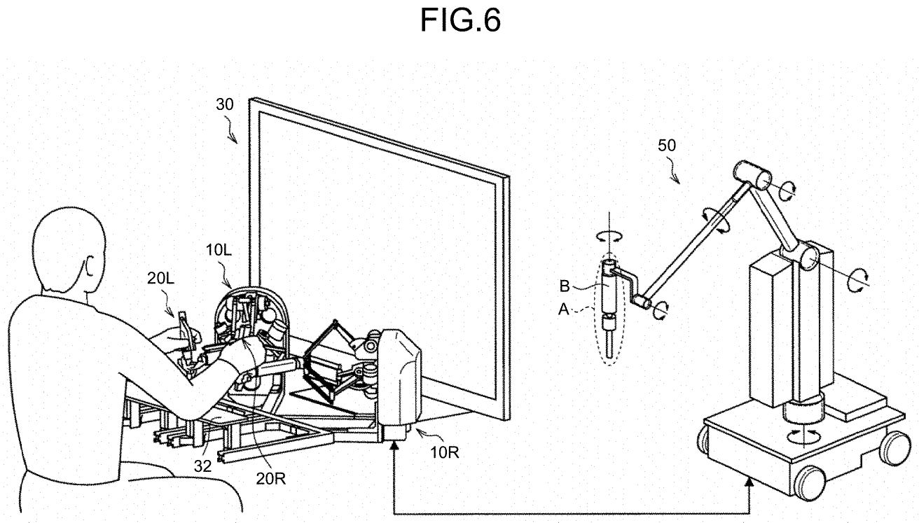

Furthermore, the present disclosure may be a master-slave device as illustrated in FIG. 6. FIG. 6 is a diagram illustrating an example of a configuration of a master-slave device according to the embodiment of the present disclosure.

A master device 10 is an information processing device (first information processing device) having a function of performing drive control of a slave device 50 and presenting a vibration signal (first signal) measured by a sensor of the slave device 50 to the user. The master device 10 is a device (a device having a link mechanism including a passive joint) having one or more joints including a passive joint and a link connected to the joint. Note that the passive joint is a joint that is not driven by a motor, an actuator, or the like.

As illustrated in FIG. 6, the master device 10 includes an operation device 20 (20R, and 20L) gripped and operated by the user. The operation device 20 corresponds to a tactile perception presenting device according to the embodiment of the present disclosure. Furthermore, the master device 10 is connected to a monitor 30 that displays a surgical field, and is provided with a support base 32 on which the user places their both arms or both elbows. The master device 10 includes a right-hand master device 10R and a left-hand master device 10L. Furthermore, the right-hand master device 10R includes a right-hand operation device 20R, and the left-hand master device 10L includes a left-hand operation device 20L.

The user places their arms or elbows on the support base 32, and grips the operation devices 20R and 20L with the right hand and the left hand, respectively. In this state, the user operates the operation devices 20R and 20L while viewing the monitor 30 on which the surgical field is displayed. By displacing the position and the direction of each of the operation devices 20R and 20L, the user may remotely operate the position or the orientation of the surgical tool attached to the slave device 50 or perform the gripping operation by each of the surgical tools.

The slave device 50 may be an information processing device (second information processing device) that presents, to the master device 10, force and vibration generated when an affected part (hereinafter also referred to as a target object) of a patient in surgery comes into contact with a part of the slave device 50 that comes in contact with the target object. The slave device 50 is, for example, a device (a device having a link mechanism including an active joint) having one or more active joints and a link connected to the active joint, provided to allow movement corresponding to the movement of the master device 10. Note that the active joint is a joint driven by a motor, an actuator, or the like.

In the slave device 50, various sensors (for example, an origin sensor, a Limit sensor, an encoder, a microphone, an acceleration sensor, or the like) are provided at the distal end portion of the arm illustrated in FIG. 6 (symbol A illustrated in FIG. 6). Furthermore, a force sensor (symbol B illustrated in FIG. 6) is provided at a distal end portion of the arm of the slave device 50. The force sensor measures force applied to the distal end portion of the arm when the distal end portion of the arm comes into contact with the patient. Note that a position where the above-described various sensors are provided is not particularly limited, and the various sensors may be provided at any position of the distal end portion of the arm.

The slave device 50 includes, for example, a motion sensor for measuring the motion of the active joint at a position corresponding to each of the active joints. Examples of the motion sensor include an encoder. Furthermore, the slave device 50 includes, for example, drive mechanisms for driving the active joints at a position corresponding to each of the active joints. Examples of the drive mechanism include a motor and a driver.

Note that the embodiments of the present disclosure may be applied to a virtual reality environment. For example, when the master device 10 is operated, a video image indicating a virtual environment on the side of the slave device 50 may be projected on the monitor 30, and the user may operate the master device 10 based on the video image.

[4. Medical Observation System]

[4-1. Configuration of Medical Observation System]

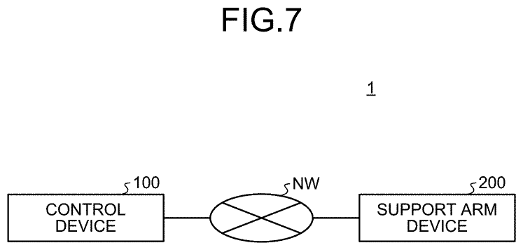

A configuration of a medical observation system according to the embodiment of the present disclosure will be described with reference to FIG. 7. FIG. 7 is a diagram illustrating an example of a configuration of a medical observation system according to the embodiment of the present disclosure.

As illustrated in FIG. 7, a medical observation system 1 according to the embodiment of the present disclosure includes a control device 100 and a support arm device 200. The control device 100 and the support arm device 200 are communicably connected via a network NW.

The medical observation system 1 determines a sensor to be used for measuring the internal environment of the patient to be used to generate an environment map indicating the patient's internal map information among a plurality of types of sensors provided in the support arm device 200 or in the medical observation device (endoscope or the like) held by the support arm. The medical observation system 1 generates an environment map indicating internal map information based on measurement results of an internal environment of the patient obtained by a plurality of sensor units provided in the support arm device 200 or in the medical observation device (endoscope or the like) held by the support arm.

(4-2. Endoscope Device)

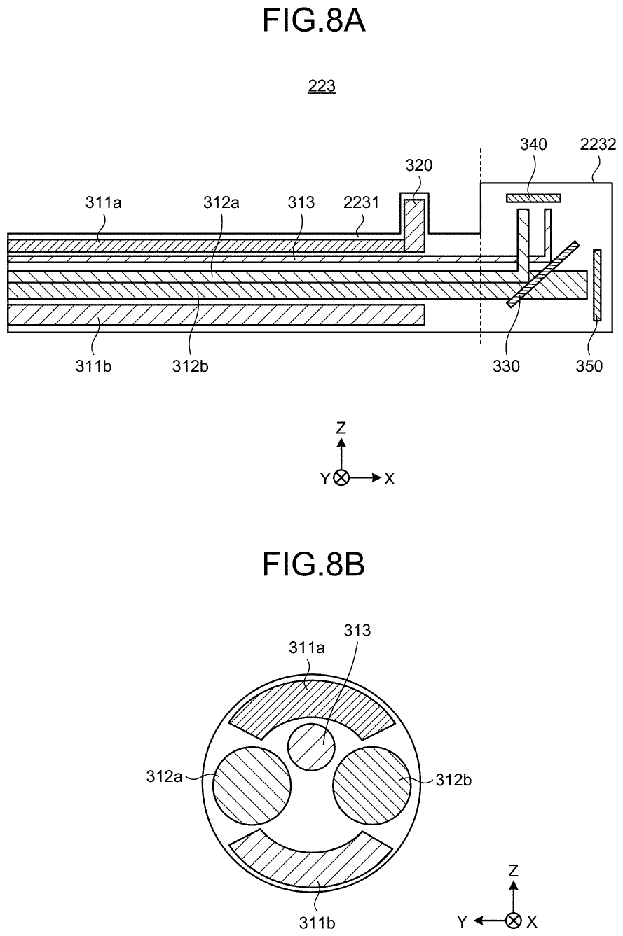

A configuration of an endoscope device according to the embodiment of the present disclosure will be described with reference to FIGS. 8A and 8B. FIG. 8A is a cross-sectional view of the endoscope device according to the embodiment of the present disclosure. FIG. 8B is a front view of a distal end portion of the endoscope device according to the embodiment of the present disclosure.

As illustrated in FIGS. 8A and 8B, the endoscope device 223 is formed with a cylindrical portion 2231 and a head portion 2232 (camera head). The endoscope device 223 includes a light guide 311a, a light guide 311b, a lens 312a, a lens 312b, an irradiation unit 313, a light source adapter 320, a half mirror 330, a sensor unit 340, and an image sensor 350.

The light guide 311a and the light guide 311b irradiate a measurement target object (for example, an organ of a patient) with light. The light guide 311a and the light guide 311b are connected to a light source device (not illustrated) via the light source adapter 320 and a light guide cable. The light guide 311a and the light guide 311b irradiate the measurement target object with light from the light source device. The light guide 311a is provided in an upper part of the cylindrical portion 2231. The light guide 311b is provided at a lower part of the cylindrical portion 2231. The light guide 311a and the light guide 311b can be formed with an optical fiber, for example.

The lens 312a and the lens 312b are optical systems to focus incident light. Part of the light focused by the lens 312a and the lens 312b is reflected by the half mirror 330 and reaches the sensor unit 340. Part of the light focused by the lens 312a and the lens 312b is transmitted through the half mirror 330 and reaches the image sensor 350. The lens 312a is provided on the left-hand side of the cylindrical portion 2231. The lens 312b is provided at the right-hand side of the cylindrical portion 2231. With this configuration, the lens 312a and the lens 312b constitute a stereo camera. Note that the endoscope is a compound eye type (stereo), but may be a monocular type.

The irradiation unit 313 is connected to the sensor unit 340. The irradiation unit 313 outputs, to the target object, light or sound for measuring the distance from the distal end portion of the cylindrical portion 2231 to the target object.

The sensor unit 340 measures various types of information regarding the target object. The sensor unit 340 is preferably a sensor capable of acquiring, for example, distance information from the distal end portion of the cylindrical portion 2231 to the target object and shape information including the target object or its peripheral portions. The sensor unit 340 may include a plurality of sensors instead of a single sensor. The sensor unit 340 can include, for example, two or more types of sensors. In the present disclosure, an internal environment map is generated based on measurement results of a plurality of types of sensors constituting the sensor unit 340.

An example of the sensor unit 340 is a stereo image sensor that calculates the distance to the target object by a triangulation method using the lens 312a and the lens 312b. The sensor unit 340 may be, for example, a Time of Flight (ToF) sensor that emits light toward the target object and calculates a distance to the target object based on the time until the emitted light is returned from the target object on which the emitted light is reflected. In this case, for example, it is possible to acquire distance (depth) information for each of pixels of the image sensor 350 that detects the reflected light, leading to achievement of construction of three-dimensional space information having a relatively high resolution.

The sensor unit 340 may be, for example, a phase difference sensor that calculates the distance to the target object based on a difference between a phase of light emitted to the target object and a phase of the reflected light from the target object. The sensor unit 340 may be, for example, an ultrasonic sensor that emits a sound wave to the target object and calculates the distance to the target object based on the time until the emitted sound wave is reflected by the object and returned.

The sensor unit 340 may be, for example, a sensor that constructs three-dimensional space information regarding the target object. The sensor unit 340 may be, for example, a sensor that irradiates the target object with pattern light, images the target object with a stereo camera, and constructs three-dimensional space information regarding the target object based on the shape of the imaged pattern light. In this method, for example, it is possible to reconstruct three-dimensional space information even in a situation where an object having a smaller change in an image is set as an imaging target. Specifically, in imaging an organ or the like of a patient, there is an assumable case where the captured portion has few irregularities, making it difficult to distinguish details of the shape from the image. In such a case, irradiating the pattern light would make it easy to distinguish the details of the shape, facilitating reconstruction of three-dimensional space information with improved accuracy.

The sensor unit 340 may be a polarization image sensor. The polarization image sensor is an image sensor capable of detecting only a part of polarized light among various types of polarized light included in incoming light. By reconstructing the three-dimensional space using the image captured by such a polarization image sensor, it is possible to generate or update the environment map. The use of this method would make it possible to prevent degradation in accuracy related to reconstruction of a three-dimensional space due to occurrence of a phenomenon referred to as blown-out highlights or overexposure due to an excessive amount of light. Furthermore, as another example, the use of this method also makes it possible to more stably reconstruct a three-dimensional space of an environment in the presence of transparent or translucent objects (for example, body tissue) or objects having different polarization degrees that are difficult to recognize with the naked eye. Furthermore, by using this method, for example, even in a situation having visible noise in a captured image or decreased contrast in the captured image due to occurrence of mist with use of an electric scalpel or the like, it is still possible to reduce the influence of the mist.

The image sensor 350 is an imaging element, and is formed with a CMOS, for example. The beams of light incident on the lens 312a and the lens 312b are focused on the image sensor 350, thereby forming an image of the target object.

The following will describe a case where the sensor unit 340 is provided in the endoscope device 223 to generate an environment map indicating information (three-dimensional information or the like) of the internal environment of the human. The present disclosure, however, is not limited this description. For example, the present disclosure may be applied to a case where the sensor unit 340 is provided in a surgical field camera installed in an operating room. Furthermore, the present disclosure may also be applied to a case where the sensor unit 340 is provided in an exoscope or a surgical microscope. In this case, the surgical field camera, the exoscope, or the surgical microscope is only required to capture at least the state of the surgical site of the patient in the operating room. Furthermore, in the present disclosure, the sensor unit 340 may be provided at the distal end portion of the arm of the slave device 50 as illustrated in FIG. 6.

(4-3. Sensor Unit)

Next, the sensor unit 340 will be described.

In the present disclosure, the environment map is generated based on the measurement result regarding the internal target object of the patient, obtained by the sensor unit 340. However, there are assumable factors, in the human during surgery, that would deteriorate the reliability of the measurement result of the sensor unit 340.

For example, there is an assumable case where parts such as the lens 312a, the lens 312b, or the irradiation unit 313 is contaminated within the patient. The contamination is caused by, for example, adhesion of blood due to bleeding, adhesion of liquid such as physiological saline for washing the abdominal cavity, adhesion of tissue and lipid occurring with the use of a tool such as an electric scalpel, adhesion of contamination due to contact with an organ, or the like.

In addition, changes in the environment of the internal space of the patient might influence the measurement result. Examples of these include bleeding from an organ. These also may include occurrence of mist by use of an instrument such as an electric scalpel. Furthermore, for example, the changes also might include reflection of light from an internally disposed instrument, reflection of light from an internal liquid, and reflection of light from a transparent organ. In addition, occurrence of a shadow by an internal instrument can be included.

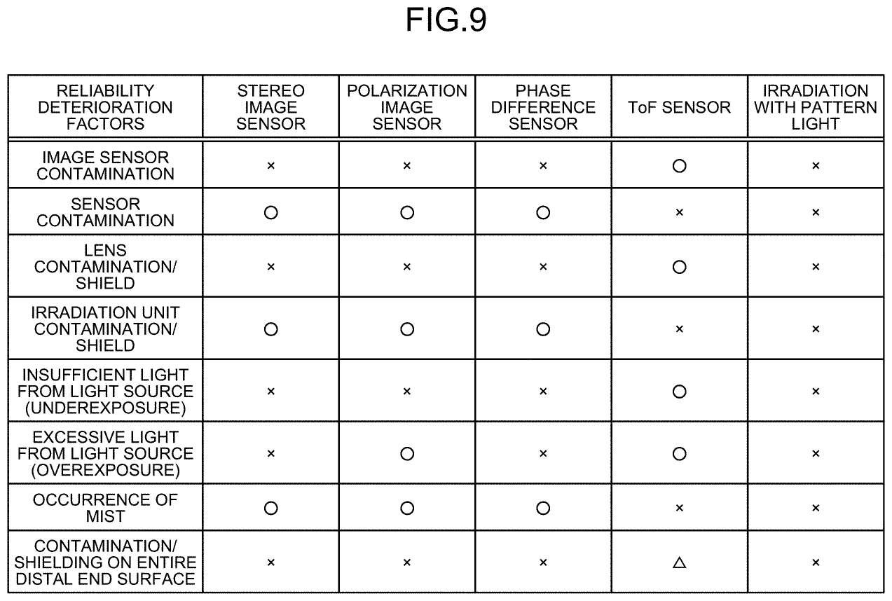

As described above, the internal environment has many factors that would deteriorate the reliability of acquired data of various sensors. With reference to FIG. 9, the reliability deterioration factors in various sensors will be described. FIG. 9 is a table illustrating robustness against reliability deterioration factors in various sensors.

In the table illustrated in FIG. 9, the symbol “∘” means that the reliability is maintained at a high level, while the symbol “x” means that the reliability is lowered. In addition, the symbol “Δ” (open triangle) means that the sensor continues to output a constant value.

FIG. 9 lists possible reliability deterioration factors such as “image sensor contamination”, “sensor contamination”, “lens contamination/shielding”, “irradiation unit contamination/shielding”, “insufficient light from light source (underexposure)”, “excessive light from light source (overexposure)”, “occurrence of mist”, and” contamination/shielding on entire distal end surface”. Examples of the method of acquiring the distance to the target object or the shape of the target object include “stereo image sensor”, “polarization image sensor”, “phase difference sensor”, “ToF sensor”, and “irradiation with pattern light”.

As illustrated in FIG. 9, various sensors and methods have their factors of reliability deterioration which are different from each other. In view of this, in the present disclosure, the internal environment is measured with a plurality of types of sensors in generation of the internal environment map so as to ensure the robustness against individual factors. In this case, a judgment unit 133 may make a judgment on the reliability deterioration factors based on the result of reliability determination for each of sensors.

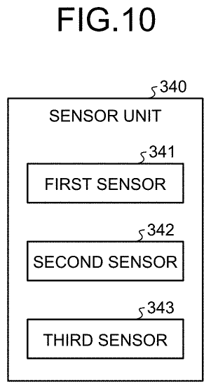

An example of a configuration of the sensor unit 340 will be described with reference to FIG. 10. FIG. 10 is a block diagram illustrating an example of a configuration of the sensor unit 340.

As illustrated in FIG. 10, the sensor unit 340 includes a first sensor 341, a second sensor 342, and a third sensor 343.

The first sensor 341 to the third sensor 343 are sensors of different types. For example, while the first sensor 341 is a stereo image sensor, the second sensor 342 is a polarization image sensor, and the third sensor 343 is a ToF sensor, the present disclosure is not limited thereto.

Hereinafter, the sensor unit 340 will be described as including three types of sensors, namely, the first sensor 341 to the third sensor 343, but the present disclosure is not limited thereto. Specifically, the sensor unit 340 is only required to include two or more types of sensors.

The control device 100 of the medical observation system 1 acquires, from the first sensor 341 to the third sensor 343, measurement results on the information regarding the distance to the internal target object of the patient or on the shape information regarding the target object. Comparison or judgment of the reliability of the measurement result is performed based on the measurement result of the internal target object of the patient obtained by the first sensor 341 to the third sensor 343. The control device 100 then determines a sensor having high reliability among the first sensor 341 to the third sensor 343. With this operation, the control device 100 can generate an environment map having high reliability.

(4-4. Control Device)

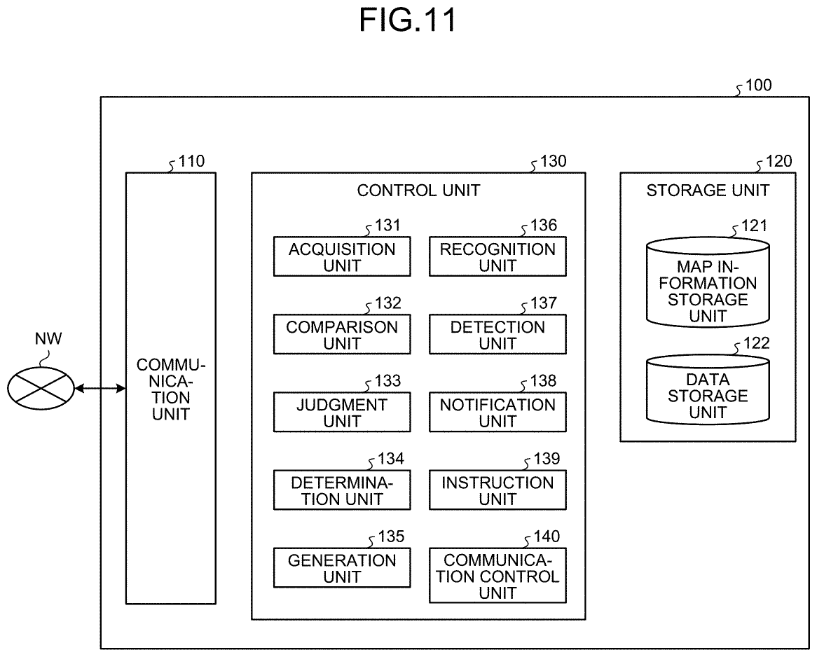

A configuration of the control device 100 will described with reference to FIG. 11. FIG. 11 is a block diagram illustrating an example of a configuration of the control device 100.

As illustrated in FIG. 11, the control device 100 includes a communication unit 110, a storage unit 120, and a control unit 130.

The communication unit 110 is actualized by, for example, a Network Interface Card (NIC), a communication circuit, or the like. The communication unit 110 is connected to a network NW (the Internet or the like) with wired or wireless communication. The communication unit 110 transmits and receives information to and from other devices or the like under the control of a communication control unit 140 via the network NW. The communication unit 110 transmits and receives information to and from the support arm device 200, for example.

The storage unit 120 is implemented by semiconductor memory elements such as Random Access Memory (RAM) and a flash drive referred to as flash memory, or other storage devices such as a hard disk or an optical disc. The storage unit 120 includes a map information storage unit 121 and a data storage unit 122.

The map information storage unit 121 stores map information indicating the internal environment of the patient. The map information may be, for example, information generated based on at least one of magnetic resonance imaging (MRI) or computed tomography (CT) before surgery on the patient. In addition, the map information may be information generated and recorded by internally observing the patient in an uncontaminated situation by a medical observation device before internal treatment is started, for example. Furthermore, the map information storage unit 121 may store the map which originally is the environment map generated before surgery or before the start of treatment and which has been sequentially updated during the surgery.

The data storage unit 122 stores various data.

The control unit 130 is actualized by execution of programs stored in the control device 100 (for example, information processing program according to the present disclosure) by a central processing unit (CPU), a micro processing unit (MPU), or the like, using random access memory (RAM) or the like, as a working area. In addition, the control unit 130 is a controller and may be implemented by an integrated circuit such as an application specific integrated circuit (ASIC) or a field programmable gate array (FPGA). The control unit 130 includes an acquisition unit 131, a comparison unit 132, a judgment unit 133, a determination unit 134, a generation unit 135, a recognition unit 136, a notification unit 138, an instruction unit 139, and a communication control unit 140.

The acquisition unit 131 acquires various types of information. The acquisition unit 131 acquires various types of information from the sensor unit 340, for example. The acquisition unit 131 acquires a measurement result regarding an internal target object of the patient or a peripheral portion thereof from the sensor unit 340. In this case, the acquisition unit 131 acquires measurement results individually from the first sensor 341 to the third sensor 343 included in the sensor unit 340.

For example, the acquisition unit 131 may have a function, in a case where a stereo image is acquired, to calculate the distance to a target object based on the stereo image.

The comparison unit 132 compares various types of information. The comparison unit 132 compares, for example, pieces of information acquired by the acquisition unit 131. For example, the comparison unit 132 compares the individual measurement results of the first sensor 341 to the third sensor 343 acquired by the acquisition unit 131.

The judgment unit 133 makes a judgment on various types of information. The judgment unit 133 makes a judgment, for example, on various types of information acquired by the acquisition unit 131. For example, the judgment unit 133 makes a judgment on the reliability of the measurement results of the first sensor 341 to the third sensor 343 acquired by the acquisition unit 131. For example, based on patient-related map information stored in the map information storage unit 121, the judgment unit 133 makes a judgment on the reliability of the measurement results of the first sensor 341 to the third sensor 343 acquired by the acquisition unit 131.

The determination unit 134 determines various types of information. For example, the determination unit 134 determines various types of information based on the comparison result of the comparison unit 132. For example, the determination unit 134 determines a sensor to be used for internal measurement of the patient among the first sensor 341 to the third sensor 343 based on a result of comparison of measurement results of the first sensor 341 to the third sensor 343 by the comparison unit 132.

The determination unit 134 determines various types of information based on the judgment result from the judgment unit 133. For example, the determination unit 134 determines a sensor to be used for internal measurement of the patient among the first sensor 341 to the third sensor 343 based on a result of judgment of the reliability in the measurement result of the first sensor 341 to the third sensor 343 by the judgment unit 133. For example, the determination unit 134 may determine two types of sensors having high reliability among the first sensor 341 to the third sensor 343 as sensors to be used for internal measurement of the patient. For example, the determination unit 134 may determine a sensor (or sensors) judged to have highest reliability among the first sensor 341 to the third sensor 343 as sensors used for internal measurement of the patient.

The generation unit 135 generates various types of information. The generation unit 135 generates various types of information based on the information determined by the determination unit 134. As an example, the generation unit 135 generates an environment map indicating the internal information of the patient based on the measurement result of the target object by the sensor determined by the determination unit 134.

The recognition unit 136 recognizes various types of information. The recognition unit 136 recognizes various types of information based on the information from the sensor unit 340 acquired by the acquisition unit 131, for example. The recognition unit 136 recognizes various types of information based on an internal video image of the patient acquired by the acquisition unit 131 from the stereo image sensor included in the sensor unit 340, for example. In this case, the recognition unit 136 recognizes bleeding, occurrence of mist, or the like generated inside the patient.

A detection unit 137 detects various types of information. The detection unit 137 detects various types of information based on a determination result by the judgment unit 133, for example. For example, the detection unit 137 detects a failure or reliability deterioration in part of the sensors based on the judgment result obtained by the judgment unit 133 regarding the reliability of the measurement results obtained by the first sensor 341 to the third sensor 343. For example, the detection unit 137 detects reliability deterioration in the entire medical observation system 1 based on the judgment result obtained by the judgment unit 133 regarding the reliability of the measurement results obtained by the first sensor 341 to the third sensor 343.

The notification unit 138 notifies various types of information. For example, the notification unit 138 notifies various types of information based on a detection result obtained by the detection unit 137. For example, when the detection unit 137 has detected reliability deterioration in the entire medical observation system 1, the notification unit 138 notifies that the environment map generated by the generation unit 135 is not in the normal state. In other words, the notification unit 138 notifies the reliability deterioration in the environment map through the user interface. In this case, the notification unit 138 may make the notification by voice using a speaker, or may make the notification by displaying a video image on the display unit.

The instruction unit 139 gives instructions of various types of information. For example, the instruction unit 139 gives instructions of various types of information based on a detection result obtained by the detection unit 137. For example, when the detection unit 137 has detected reliability deterioration in the entire medical observation system 1, the instruction unit 139 performs various instructions to a surgical robot that performs autonomous/semi-autonomous driving based on the environment map generated by the generation unit 135. In this case, the instruction unit 139 gives an instruction of operation of the surgical robot based on the environment map, for example. More specifically, the instruction unit 139 causes the surgical robot to execute crisis avoidance operation, for example. The crisis avoidance operation includes, for example, stop of treatment, removal operation of a medical instrument inserted into a patient, or the like.

The communication control unit 140 controls transmission and reception of information via the communication unit 110. The communication control unit 140 controls the communication unit 110 to communicate with other information processing device(s). For example, the communication control unit 140 controls the communication unit 110 to communicate with the support arm device 200.

For example, the communication control unit 140 controls the communication unit 110 to communicate with the surgical robot.

[5. Processes of Medical Observation System]

(5-1. Outline of Processes of Medical Observation System)

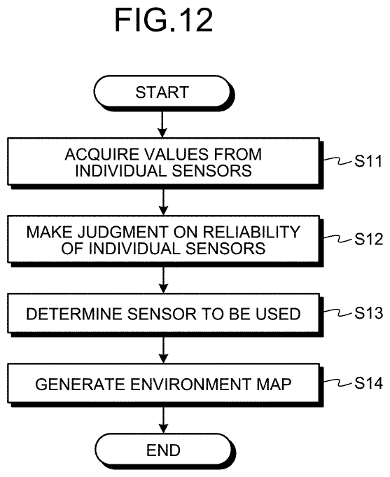

Processes of the medical observation system 1 will be described with reference to FIG. 12. FIG. 12 is a flowchart illustrating an example of a process flow performed by the medical observation system 1.

First, the control device 100 acquires the sensor values from each of sensors included in the support arm device 200 (step S11). Subsequently, the process proceeds to step S12.

The control device 100 makes a judgment on the reliability of each of the sensors based on the acquired value of each of the sensors (step S12). Subsequently, the process proceeds to step S13.

Based on the reliability of each of the sensors, the control device 100 determines a sensor to be used for generating an internal environment map of the patient, among the sensors (step S13). Subsequently, the process proceeds to step S14.

The control device 100 generates an internal environment map of the patient using the sensor values determined in step S13 (step S14).

(5-2. First Process)

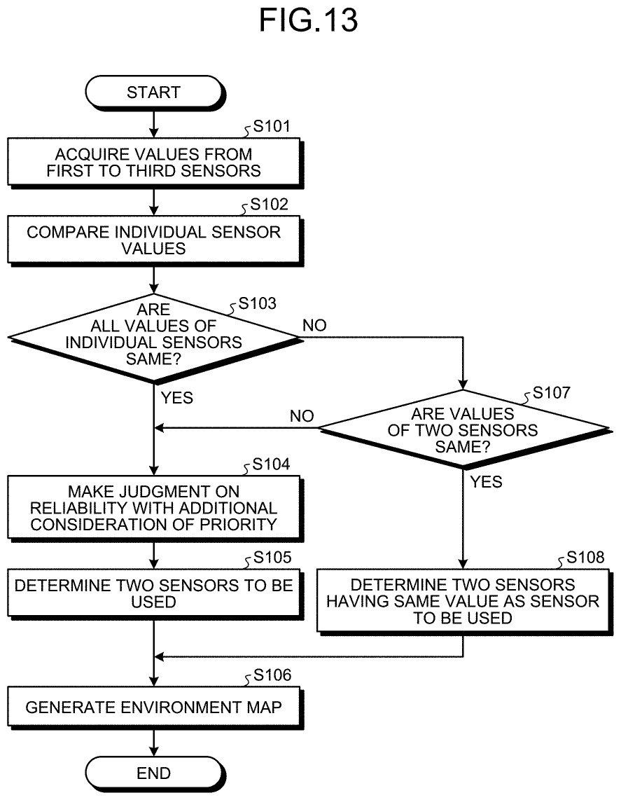

A flow of a first process of the control unit 130 of the control device 100 according to the embodiment will be described with reference to FIG. 13. FIG. 13 is a flowchart illustrating an example of a flow of the first process of the control unit 130 according to the embodiment. Hereinafter, an environment map is generated based on the values of the first sensor 341 to the third sensor 343 illustrated in FIG. 10.

First, the control unit 130 acquires sensor values from each of the first sensor 341 to the third sensor 343 (step S101). Specifically, the acquisition unit 131 acquires the sensor value from each of the first sensor 341 to the third sensor 343 via the communication unit 110. Subsequently, the process proceeds to step S102.

The control unit 130 compares the sensor values acquired from the first sensor 341 to the third sensor 343 (step S102). Specifically, the comparison unit 132 compares the sensor values acquired from the first sensor 341 to the third sensor 343. Subsequently, the process proceeds to step S103.

The control unit 130 judges whether the values of all the sensors are the same (step S103). Specifically, the judgment unit 133 judges whether the values of the sensors are all the same, thereby judging the reliability of the values of the sensors. Note that “the values of the sensors are the same” includes not only completely matching but also a case where the values fall within a predetermined range. In other words, a case where the values of the sensors are similar to each other is also included. More specifically, since the sensors perform distance measurement or the like by different methods, the values of the sensors are expected to be different from each other. In such a case, the judgment unit 133 judges whether data derived from sensor values measured by different types of sensors is the same or falls within a predetermined range. When it is judged that all the values of the sensors are the same (Yes in step S103), the process proceeds to step S104. When it is judged that all the values of the sensors are not the same (No in step S103), the process proceeds to step S107.

When the judgment is Yes in step S103, the control unit 130 makes a judgment on the reliability of the sensor with additional consideration of a predetermined priority to each of sensors (step S104). Specifically, the judgment unit 133 makes a judgment on the reliability of each of sensors, namely, the first sensor 341 to the third sensor 343 with additional consideration of a predetermined priority. For example, since a stereo image sensor that actually captures internal video images is expected to be mainly used with high possibility, the highest priority may be assigned to the stereo image sensor. Subsequently, the process proceeds to step S105.

The control unit 130 determines two sensors to be used for generating the environment map based on the judgment result of step S104 (step S105). Specifically, the determination unit 134 determines two sensors with high reliability among the first sensor 341 to the third sensor 343. Subsequently, the process proceeds to step S106.

The control unit 130 generates an internal environment map of the patient using the values of the two sensors determined in step S105 (step S106). Specifically, the generation unit 135 generates an internal environment map of the patient using the values of the two sensors determined among the first sensor 341 to the third sensor 343. This completes the process of FIG. 13.

As another course of steps, when it is judged as No in step S103, the control unit 130 judges whether the values of the two sensors, among the values of the individual sensors, are the same (step S107). Specifically, the judgment unit 133 judges whether the values of the two sensors, among the values of the individual sensors, are the same, thereby making a judgment on the reliability. In this case, the judgment unit 133 judges that two sensors having the same value have high reliability while the one sensor having a different value has low reliability. When it is judged that the values of the two sensors are not the same, that is, the values of the respective sensors are all different from each other (No in step S107), the process proceeds to step S104. When the values of all the sensors are different, the judgment unit 133 judges that the reliability of the entire medical observation system 1 is low, for example. As another course of steps, when it is judged that the values of the two sensors are the same (Yes in step S107), the process proceeds to step S108.

When it is determined as Yes in step S107, the control unit 130 determines two sensors having the same value as the sensors to be used for generating the internal environment map of the patient (step S108). Specifically, the determination unit 134 determines two sensors having the same value among the first sensor 341 to the third sensor 343 as sensors for generating an internal environment map of the patient. This completes the process of FIG. 13.

As described above, in the present embodiment, the reliability of individual sensors can be judged based on the value of the sensors. The present embodiment can then determine a sensor (or sensors) for generating an internal environmental map of the patient based on the reliability. With this configuration, the present embodiment can improve the accuracy of the environment map.

(5-3. Second Process)

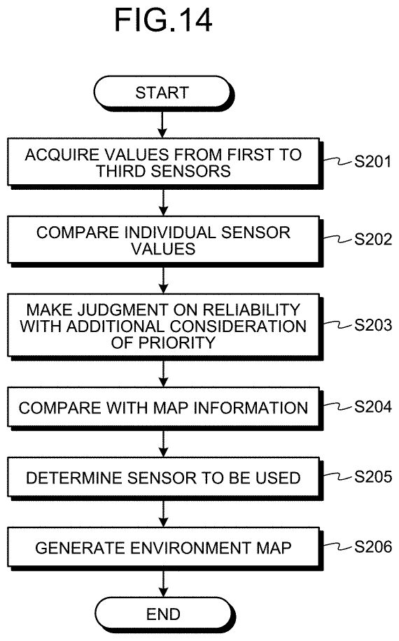

A flow of a second process of the control unit 130 of the control device 100 according to the embodiment will be described with reference to FIG. 14. FIG. 14 is a flowchart illustrating an example of a flow of the second process of the control unit 130 according to the embodiment.

Step S201, step S202, and step S203 are the same as step S101, step S102, and step S104 illustrated in FIG. 13, respectively, and thus, description thereof is omitted.

The control unit 130 compares the value of each of sensors with the map information of the patient stored in the map information storage unit 121 (step S204). Specifically, the judgment unit 133 compares the values of the first sensor 341 to the third sensor 343 with the map information stored in the map information storage unit 121 to make a judgment on the reliability of each of the first sensor 341 to the third sensor 343. In this case, a sensor having a value closer to the value of the map information has higher reliability, and a sensor having a value farther from the value of the map information has lower reliability. Subsequently, the process proceeds to step S205.

The control unit 130 determines the sensor to be used for generating the environment map based on the judgment result of step S204 (step S205). Specifically, the determination unit 134 determines the sensor having high reliability among the first sensor 341 to the third sensor 343. Here, the determination unit 134 may determine one sensor with the highest reliability or may determine two sensors with the high reliability. Subsequently, the process proceeds to step S206.

The control unit 130 generates an internal environment map of the patient using the values of the sensor determined in step S205 (step S206). Specifically, the generation unit 135 generates an internal environment map of the patient using the values of the sensor determined among the first sensor 341 to the third sensor 343. This completes the process of FIG. 14.

As described above, in the present embodiment, the reliability of each sensors can be judged based on the comparison result between the value of each of the sensors and the map information of the patient generated in advance. In addition, in the present embodiment, it is possible to determine a sensor to be used for generating the internal environment map of the patient based on the reliability judged based on the result of comparison with the map information. With this configuration, the present embodiment can further improve the accuracy of the environment map. Incidentally, map information generated before surgery based on MRI or CT imaging might be influenced by shape deformation or position displacement of organs due to changes in the abdominal cavity pressure or a change in the body posture during surgery. In view of this, it is also allowable to correct the preliminarily generated map information and use the corrected version for the comparison.

(5-4. Third Process)

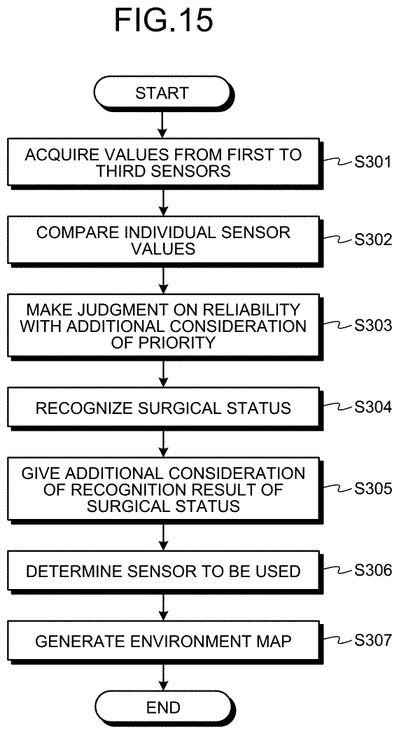

A flow of a third process of the control unit 130 of the control device 100 according to the embodiment will be described with reference to FIG. 15. FIG. 15 is a flowchart illustrating an example of a flow of the third process of the control unit 130 according to the embodiment.

Steps S301 to S303 are the same as steps S201 to S203 illustrated in FIG. 14, respectively, and thus, the description thereof is omitted.

The control unit 130 recognizes a surgical status (step S304). Specifically, the recognition unit 136 recognizes a surgical status such as occurrence of mist or bleeding based on the video image of the surgical field acquired by the acquisition unit 131. Subsequently, the process proceeds to step S305.

The control unit 130 gives additional consideration of the recognition result of the surgical status by the recognition unit 136 (step S305). Specifically, the judgment unit 133 makes a judgment on the reliability of the first sensor 341 to the third sensor 343 according to the recognition result of the occurrence of mist or the occurrence of bleeding obtained by the recognition unit 136. For example, in a case where two of the first sensor 341 to the third sensor 343 are the image sensor and the ToF sensor, and in a case where the recognition unit 136 has recognized the occurrence of mist based on the video image acquired by the image sensor, the judgment unit 133 judges that the reliability of the ToF sensor is low. That is, the judgment unit 133 can judge the change in the reliability of each of sensors according to the surgical status. Furthermore, the judgment unit 133 may judge the contamination on the sensor unit 340 based on ToF sensor values. For example, when the ToF sensor continues to output a constant value, the judgment unit 133 may judge that there is contamination in the entire distal end portion of the endoscope device 223 where the sensor unit 340 is provided. Furthermore, when the reliability of the stereo image sensor is low and the reliability of the ToF sensor is high, the judgment unit 133 may judge that the light source is insufficient. Subsequently, the process proceeds to step S306.

The control unit 130 determines a sensor to be used for generating an environment map based on the judgment result of step S305 (step S306). Specifically, the determination unit 134 determines the sensor having high reliability among the first sensor 341 to the third sensor 343. Here, the determination unit 134 may determine one sensor with the highest reliability or may determine two sensors with the high reliability. Subsequently, the process proceeds to step S307.

The control unit 130 generates an internal environment map of the patient using the values of the sensor determined in step S307 (step S307). Specifically, the generation unit 135 generates an internal environment map of the patient using the values of the sensor determined among the first sensor 341 to the third sensor 343. This completes the process of FIG. 15.

As described above, in the present embodiment, the reliability of each of sensors can be judged based on the surgical status. In addition, the present embodiment can determine a sensor (or sensors) for generating an internal environment map of the patient based on the reliability judged based on the surgical status. With this configuration, the present embodiment can further improve the accuracy of the environment map.

(5-5. Fourth Process)

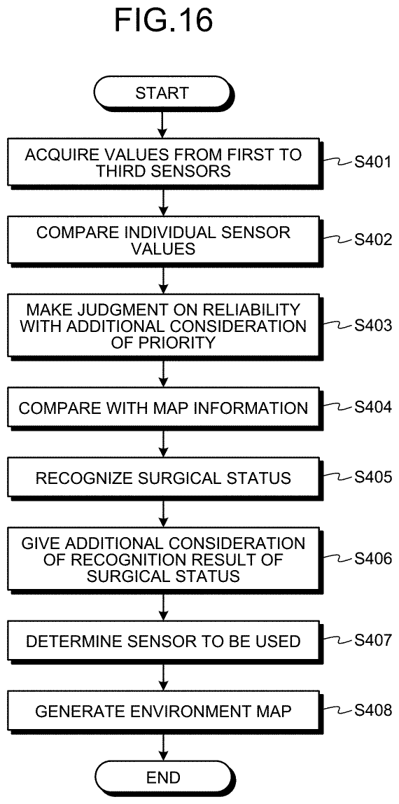

A flow of a fourth process of the control unit 130 of the control device 100 according to the embodiment will be described with reference to FIG. 16. FIG. 16 is a flowchart illustrating an example of a flow of the fourth process of the control unit 130 according to the embodiment.

Steps S401 to S404 are the same as steps S201 to S204 illustrated in FIG. 14, respectively, and thus description thereof is omitted. In addition, steps S405 and S406 are the same as steps S304 and S305 illustrated in FIG. 15, respectively, and thus description thereof is omitted. That is, the fourth embodiment is a combination of the second embodiment and the third embodiment.

The control unit 130 determines a sensor to be used for generating the environment map based on the comparison result in step S404 and the judgment result in step S406 (step S407). Specifically, the determination unit 134 determines the sensor having high reliability among the first sensor 341 to the third sensor 343. Here, the determination unit 134 may determine one sensor with the highest reliability or may determine two sensors with the high reliability. Subsequently, the process proceeds to step S408.

The control unit 130 generates an internal environment map of the patient using the values of the sensor determined in step S407 (step S408). Specifically, the generation unit 135 generates an internal environment map of the patient using the values of the sensor determined among the first sensor 341 to the third sensor 343. This completes the process of FIG. 16.

As described above, in the present embodiment, the reliability of each sensors can be judged based on the comparison result between the value of each of the sensors and the map information of the patient generated in advance, and based on the surgical status. In addition, in the present embodiment, it is possible to determine a sensor for generating the internal environment map of the patient based on the result of comparison with the map information and based on the reliability judged based on the surgical status. With this configuration, the present embodiment can further improve the accuracy of the environment map.

[6. Modification of Medical Observation System]

(6-1. Configuration of Modification of Medical Observation System)

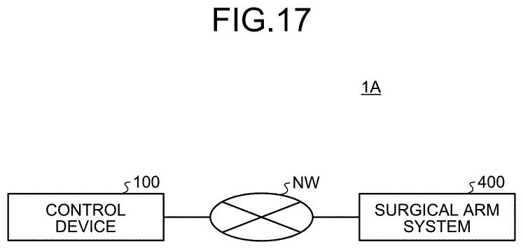

A configuration of a modification of the medical observation system according to the embodiment of the present disclosure will be described with reference to FIG. 17. FIG. 17 is a diagram illustrating a configuration of a modification of the medical observation system according to the embodiment of the present disclosure.

As illustrated in FIG. 17, a medical observation system 1A includes a control device 100 and a surgical arm system (surgical robot system) 400. The control device 100 and the surgical arm system 400 are communicably connected via a network NW. The surgical arm system 400 is a robot that is autonomously/semi-autonomously driven to perform various operations on a patient in cooperation with a surgeon. In the present embodiment, the control device 100 controls the surgical arm system 400 based on the reliability of individual sensors.

(6-2. Surgical Arm System)

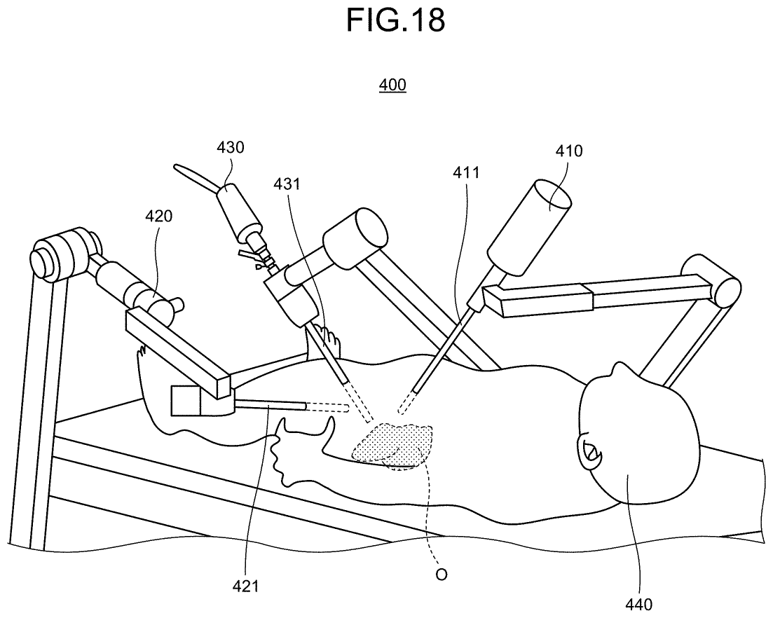

An example of a configuration of a surgical arm system will be described with reference to FIG. 18. FIG. 18 is a diagram illustrating an example of a configuration of a surgical arm system.

As illustrated in FIG. 18, the surgical arm system 400 includes, for example, a first support arm device 410, a second support arm device 420, and a third support arm device 430. The first support arm device 410 is provided with a first medical instrument 411. The second support arm device 420 is provided with a second medical instrument 421. The third support arm device 430 is provided with a third medical instrument 431. The first support arm device 410 to the third support arm device 430 have configurations similar to the configurations of the support arm device 200 illustrated in FIG. 3, but the present disclosure is not limited thereto. For example, the first support arm device 410 to the third support arm device 430 are not particularly limited as long as they can support the first medical instrument 411 to the third medical instrument 431, respectively. Furthermore, the surgical arm system 400 may further include another support arm device.

Using the first medical instrument 411 and the second medical instrument 421, the surgical arm system 400 performs various operations on a patient 440 in cooperation with a doctor (or a team including a surgeon and support workers). The third medical instrument 431 is, for example, an endoscope device and captures an internal image of the patient 440. In addition, the third medical instrument 431 is provided with a plurality of types of sensors. For example, the third medical instrument 431 is provided with a sensor unit 340 illustrated in FIG. 10. That is, the control device 100 calculates a distance between the distal end portion of the third medical instrument and an organ O by the sensor unit 340 provided at the distal end portion of the third medical instrument 431. By repeating this operation, an internal environment map of the patient 440 is generated. In addition, the control device 100 causes the surgical arm system 400 to execute the crisis avoidance operation based on the measurement result of the sensor unit 340 provided in the third medical instrument 431.

(6-3. Processing of Modification of Medical Observation System)

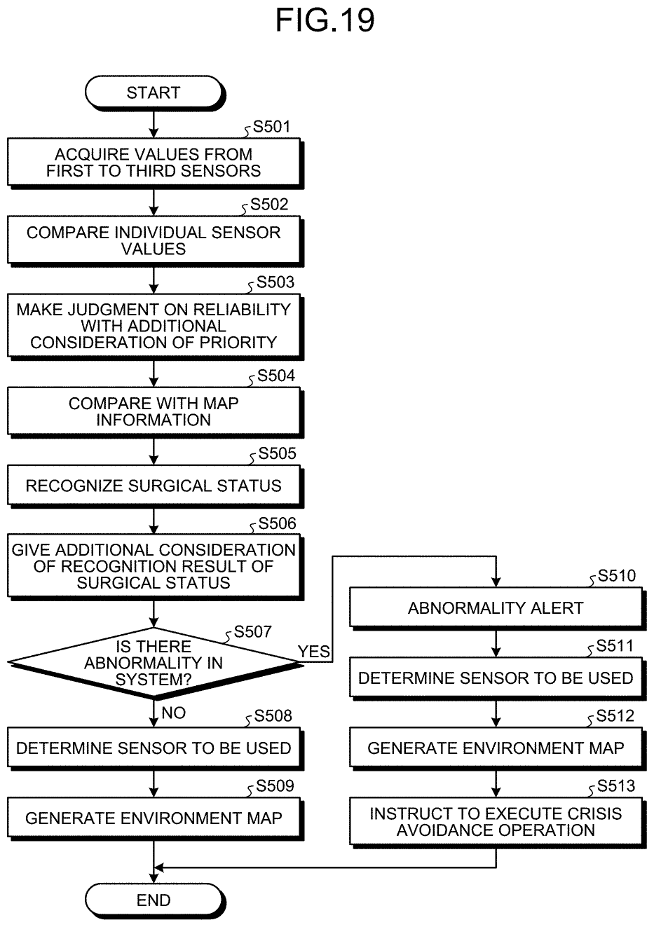

A flow of the process of the control unit 130 of the control device 100 according to a modification of the embodiment will be described with reference to FIG. 19. FIG. 19 is a flowchart illustrating an example of a flow of processes of the control unit 130 according to the modification of the embodiment.

Since steps S501 to S506 are the same as steps S401 to S406 illustrated in FIG. 16, respectively, the description thereof is omitted.

The control unit 130 judges whether there is an occurrence of abnormality in the medical observation system 1A (step S507). Specifically, when the detection unit 137 has detected a failure or deterioration in reliability of all the sensors included in the sensor unit 340, it is judged that there is an abnormality in the medical observation system 1A. When it is determined that there is no abnormality in the medical observation system 1A (No in step S507), the process proceeds to step S508. When it is determined that there is an abnormality in the medical observation system 1A (Yes in step S507), the process proceeds to step S510.

Step S508 and step S509 are the same as step S407 and step S408 illustrated in FIG. 16, respectively, and thus description thereof is omitted.

When it is judged as Yes in step S507, the control unit 130 notifies an occurrence of an abnormality in the medical observation system 1A by an abnormality alert (step S510). Specifically, the notification unit 138 notifies the occurrence of an abnormality in the medical observation system 1A by sounding an abnormality alert from a speaker (not illustrated) or the like. Subsequently, the process proceeds to step S511.

The control unit 130 determines a sensor to be used for generating an internal environment map of the patient (step S511). Specifically, the determination unit 134 determines a sensor with relatively high reliability among the first sensor 341 to the third sensor 343 with reduced reliability, as a sensor to be used for generating the environment map. Subsequently, the process proceeds to step S512.

The control unit 130 generates an internal environment map of the patient using the value of the sensor determined in step S511 (step S512). Specifically, the generation unit 135 generates the internal environment map of the patient using the value of the sensor with relatively good reliability determined by the determination unit 134. Subsequently, the process proceeds to step S513.

The control unit 130 instructs the surgical arm system 400 to perform crisis avoidance operation based on the environment map created in step S512 (step S513). Specifically, the instruction unit 139 instructs the surgical arm system 400 to stop operation on the patient. As a response to this, the surgical arm system 400 performs insertion/extraction operations on the first medical instrument 411 to the third medical instrument 431 on the patient, for example. This completes the process of FIG. 19.

As described above, according to the modification of the embodiment, it is possible to instruct the surgical arm system 400 to perform the crisis avoidance operation at the time of deterioration of the reliability of the entire medical observation system 1A. This makes it possible to improve the safety of the medical observation system 1A.

[7. Hardware Configuration]

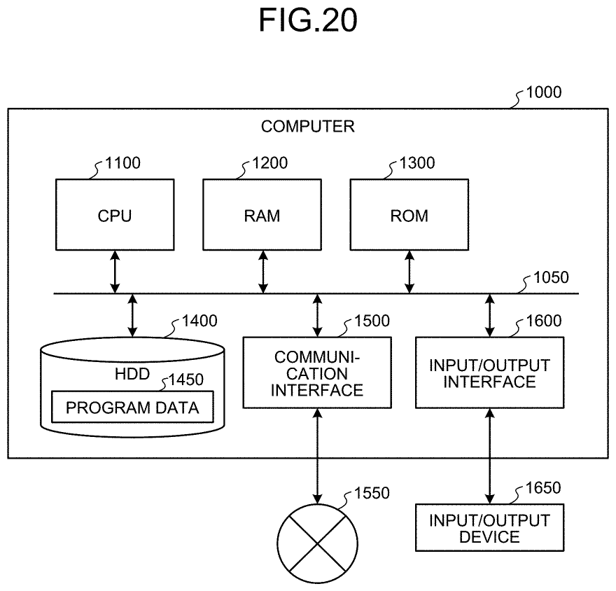

The information device such as the control device 100 described above is actualized by a computer 1000 having a configuration as illustrated in FIG. 20, for example. FIG. 20 is a hardware configuration diagram illustrating an example of the computer 1000 that implements the functions of the information processing device such as the control device 100. Hereinafter, the control device 100 according to the embodiment will be described as an example. The computer 1000 includes a CPU 1100, RAM 1200, read only memory (ROM) 1300, a hard disk drive (HDD) 1400, a communication interface 1500, and an input/output interface 1600. Individual components of the computer 1000 are interconnected by a bus 1050.

The CPU 1100 operates based on a program stored in the ROM 1300 or the HDD 1400 so as to control each of components. For example, the CPU 1100 develops a program stored in the ROM 1300 or the HDD 1400 into the RAM 1200 and executes processes corresponding to various programs.

The ROM 1300 stores a boot program such as a basic input output system (BIOS) executed by the CPU 1100 when the computer 1000 starts up, a program dependent on hardware of the computer 1000, or the like.

The HDD 1400 is a non-transitory computer-readable recording medium that records a program executed by the CPU 1100, data used by the program, or the like. Specifically, the HDD 1400 is a recording medium that records an information processing program according to the present disclosure, which is an example of program data 1450.

The communication interface 1500 is an interface for connecting the computer 1000 to an external network 1550 (for example, the Internet). For example, the CPU 1100 receives data from other devices or transmits data generated by the CPU 1100 to other devices via the communication interface 1500.

The input/output interface 1600 is an interface for connecting between an input/output device 1650 and the computer 1000. For example, the CPU 1100 receives data from an input device such as a keyboard or a mouse via the input/output interface 1600. In addition, the CPU 1100 transmits data to an output device such as a display, a speaker, or a printer via the input/output interface 1600. Furthermore, the input/output interface 1600 may function as a media interface for reading a program or the like recorded on predetermined recording medium (or simply medium). Examples of the media include optical recording media such as a digital versatile disc (DVD) or a phase change rewritable disk (PD), a magneto-optical recording medium such as a magneto-optical disk (MO), a tape medium, a magnetic recording medium, and semiconductor memory.

For example, when the computer 1000 functions as the control device 100 according to the embodiment, the CPU 1100 of the computer 1000 executes the information processing program loaded on the RAM 1200 so as to implement the functions of the control unit 130 or the like. Furthermore, the HDD 1400 stores the information processing program according to the present disclosure or data in the storage unit 14. While the CPU 1100 executes the program data 1450 read from the HDD 1400, the CPU 1100 may acquire these programs from another device via the external network 1550, as another example.

(Effects)

The medical observation system 1 includes: the plurality of types of sensor units 340 that measure information regarding an internal environment; the acquisition unit 131 that acquires individual sensor values of the plurality of types of sensor units 340; the comparison unit 132 that compares the individual sensor values of the plurality of types of sensor units 340 acquired by the acquisition unit 131; and the determination unit 134 that determines the sensor unit 340 to be used for observing the internal environment among the plurality of types of sensor units 340 based on a comparison result obtained by the comparison unit 132.

With this configuration, the sensor unit 340 to be used for observing the internal environment is determined based on the result of comparison between the individual sensor values of the plurality of types of sensor units 340. As a result, it is possible to optimize the sensor unit 340 to be used to generate the internal environment map.

The medical observation system 1 may include the generation unit 135 that generates the internal environment map based on the sensor value of the sensor unit 340 determined by the determination unit 134.

With this configuration, the environment map of the internal environment can be created with high accuracy.

The medical observation system 1 may include a judgment unit that makes a judgment on the reliability of each of the plurality of types of sensor units 340 based on the comparison result obtained by the comparison unit 132. The determination unit 134 determines a sensor unit to be used to observing the internal environment among the plurality of types of sensor units 340 based on the judgment result obtained by the judgment unit 133.

With this configuration, it is possible to further optimize the sensor unit 340 to be used to generate the internal environment map. This further improves the accuracy of the environment map.

The determination unit 134 may determine at least two types of sensor units 340 having high reliability as sensor units to be used for observing the internal environment based on the judgment result obtained by the judgment unit 133.

With this configuration, it is possible to determine two or more types of highly reliable sensor units 340 and generate an environment map from an average of the sensor values. This further improves the accuracy of the environment map as a result.

The determination unit 134 may determine the sensor unit having the highest reliability as a sensor unit to be used for observing the internal environment based on the judgment result obtained by the judgment unit 133.

With this configuration, it is possible to determine the sensor units 340 having the highest reliability and generate an environment map only from the value of the sensor unit 340. This makes it possible to generate the environment map without including the sensor values of the other sensor units 340 having low reliability, leading to the improved accuracy of the environment map.

The judgment unit 133 may determine the reliability of each of the plurality of types of sensor units 340 based on the priority set in advance for each of the plurality of types of sensor units 340.

With this configuration, the environment map can be generated using the sensor value of the sensor unit 340 having the highest robustness as a main sensor. This makes it possible to generate an environment map without including the sensor values of the sensor unit 340 having the lowest reliability under the environment where the reliability of all the sensor units 340 is lowered, leading to further improvement of accuracy of the environment map.

One of the plurality of types of sensor units 340 may be an image sensor that images the internal environment. The medical observation system 1 includes the recognition unit 136 that recognizes the status based on the video image acquired from the image sensor. The judgment unit 133 may make a judgment on the reliability of each of the plurality of types of sensor units 340 based on the recognition result obtained by the recognition unit 136.

With this configuration, it is possible to judge the reliability of the plurality of types of sensor units 340 with additional consideration of the recognition result of the surgical status. This further improves the accuracy of the environment map as a result.

The judgment unit 133 may make a judgment on the reliability of each of the plurality of types of sensor units 340 based on map information regarding the internal environment before surgery.

With this configuration, it is possible to make a judgment on the reliability of the plurality of types of sensor units 340 by comparing the information before surgery with the sensor values of the plurality of types of sensor units 340 during surgery. This further improves the accuracy of the environment map as a result.

The map information regarding the internal environment before surgery may be generated based on at least one of magnetic resonance imaging (MRI) or computed tomography (CT).

With this configuration, the map information regarding the internal environment before surgery can be generated using an existing device such as MRI or CT devices.

The judgment unit 133 may make a judgment on the reliability of each of the plurality of types of sensor units based on the map information regarding the internal environment before surgery and based on the environment map.

With this configuration, the reliability of the plurality of types of sensor units 340 can be judged by comparing the map information generated by the sensor unit 340 at the start of the surgery with the sensor values of the plurality of types of sensor units 340 during the surgery. This further improves the accuracy of the environment map as a result.

One of the plurality of types of sensor units 340 may be an image sensor that images the internal environment. The medical observation system 1 may include the recognition unit 136 that recognizes the status based on the video image acquired from the image sensor. The judgment unit 133 may make a judgment on the reliability of each of the plurality of types of sensor units 340 based on the priority set in advance for each of the plurality of types of sensor units 340, the recognition result obtained by the recognition unit 136, the map information regarding the internal environment before surgery, and the environment map.

With this configuration, the accuracy of the environment map is further improved.

The medical observation system 1A may include the detection unit 137 that detects a failure or deterioration in reliability of at least part of the sensor units 340 based on the judgment result obtained by the judgment unit 133.

With this configuration, the medical observation system 1 can self-judge the sensor failure and deterioration of the internal environment. This results in further improvement in safety.

The detection unit 137 may detect deterioration in the reliability of the entire medical observation system 1.

With this configuration, the medical observation system 1 can self-judge the sensor failure and deterioration of the internal environment. This results in further improvement in safety.

When the detection unit 137 has detected deterioration in the reliability of the entire medical observation system 1, the generation unit 135 may generate an environment map based on the measurement result of the sensor unit having relatively high reliability among the plurality of types of sensor units 340.

With this configuration, even in a case where the reliability of the medical observation system 1 is low, it is possible to generate the environment map for avoiding the crisis. This results in further improvement in safety.

The medical observation system 1 (1A) may include the notification unit 138, which notifies deterioration in the reliability of the environment map when the detection unit 137 has detected the deterioration in the reliability of the entire medical observation system 1 (1A).

With this configuration, when the autonomous operation is turned on, it is possible to perform autonomous notification of the deterioration in the accuracy of the environment map. This results in further improvement in safety.

The medical observation system 1(1A) may include the instruction unit 139 that gives an instruction of the operation of the autonomously driven main body in accordance with the environment map generated based on the measurement result obtained by the sensor unit 340 having relatively high reliability among the plurality of types of sensor units 340 when the detection unit 137 has detected deterioration in the reliability of the entire medical observation system 1(1A).

With this configuration, when the autonomous operation is turned on, it is possible to autonomously execute the operation in accordance with the environment map. This results in further improvement in safety.

The instruction unit 139 may cause the main body to execute the crisis avoidance operation.

With this configuration, when the autonomous operation is turned on, it is possible to execute the operation of avoiding the crisis autonomously. This results in further improvement in safety.

The plurality of types of sensor units 340 may be provided in the endoscope and may include a stereo image sensor and a Time of Flight (ToF) sensor. The judgment unit 133 may make a judgment on the deterioration in reliability of the ToF sensor due to the mist based on the recognition result obtained by the recognition unit 136.

With this configuration, it is possible to optimize the sensor unit 340 to be used to generate the environment map, in accordance with the change in the internal environment. This further improves the accuracy of the environment map as a result.

The judgment unit 133 may detect contamination on the distal end portion of the lens barrel of the endoscope based on the value of the ToF sensor.

With this configuration, it is possible to optimize the sensor unit 340 to be used to generate the environment map, in accordance with the change in the internal environment. This further improves the accuracy of the environment map as a result.

The judgment unit 133 may detect a light source defect in the imaging range of the stereo image sensor based on a result of comparison between the value of the stereo image sensor and a value of the ToF sensor.

With this configuration, it is possible to optimize the sensor unit 340 to be used to generate the environment map, in accordance with the change in the internal environment. This further improves the accuracy of the environment map as a result.

The medical observation system 1(1A) may include a support arm device having an arm unit at least a part of which is configured to be bendable and which is configured to be able to support a medical instrument. The plurality of types of sensor units 340 may be supported by the arm unit.

With this configuration, the present disclosure can be applied to a device including an arm unit.

The control device 100 includes: the acquisition unit 131 that acquires individual sensor values of the plurality of types of sensor units 340 that measure information regarding an internal environment; the comparison unit 132 that compares the individual sensor values of the plurality of types of sensor units 340 acquired by the acquisition unit 131; and the determination unit 134 that determines the sensor unit 340 to be used for observing the internal environment among the plurality of types of sensor units 340 based on a comparison result obtained by the comparison unit 132.

With this configuration, the sensor unit 340 to be used for observing the internal environment is determined based on the result of comparison between the individual sensor values of the plurality of types of sensor units 340. As a result, it is possible to optimize the sensor unit 340 to be used to generate the internal environment map.

The control method includes: acquiring individual sensor values of the plurality of types of sensor units 340 that measure information regarding an internal environment; comparing the acquired individual sensor values of the plurality of types of sensor units 340; and determining a sensor unit to be used for observing the internal environment among the plurality of types of sensor units 340 based on a comparison result.

With this configuration, the sensor unit 340 to be used for observing the internal environment is determined based on the result of comparison between the individual sensor values of the plurality of types of sensor units 340. As a result, it is possible to optimize the sensor unit 340 to be used to generate the internal environment map.

The effects described in the present specification are merely examples, and thus, there may be other effects, not limited to the exemplified effects.

Note that the present technology can also have the following configurations.

(1)

A medical observation system comprising:

a plurality of types of sensor units that measure information regarding an internal environment;

an acquisition unit that acquires individual sensor values of the plurality of types of sensor units;

a comparison unit that compares the individual sensor values of the plurality of types of sensor units acquired by the acquisition unit; and

a determination unit that determines a sensor unit to be used for observing the internal environment among the plurality of types of sensor units based on a comparison result obtained by the comparison unit.

(2)

The medical observation system according to (1), comprising a generation unit that generates an environment map of the internal environment based on a measurement result obtained by the sensor unit determined by the determination unit.

(3)

The medical observation system according to (1) or (2), comprising a judgment unit that makes a judgment on reliability of each of the plurality of types of sensor units based on the comparison result obtained by the comparison unit,

wherein the determination unit determines the sensor unit to be used for observing the internal environment among the plurality of types of sensor units based on a judgment result obtained by the judgment unit.

(4)

The medical observation system according to (3),

wherein the determination unit determines at least two types of sensor units with high reliability as the sensor units to be used for observing the internal environment based on the judgment result obtained by the judgment unit.

(5)

The medical observation system according to (3) or (4),

in which the determination unit determines a sensor unit having the highest reliability as a sensor unit to be used for observing the internal environment, based on a judgment result obtained by the judgment unit.

(6)

The medical observation system according to any one of (3) to (5),

wherein the judgment unit makes a judgment on the reliability of each of the plurality of types of sensor units based on priority set in advance for each of the plurality of types of sensor units.

(7)

The medical observation system according to any one of (3) to (6),

wherein one of the plurality of types of sensor units is an image sensor that images the internal environment,

the medical observation system further comprises a recognition unit that recognizes a status based on a video image acquired from the image sensor, and

the judgment unit makes a judgment on the reliability of each of the plurality of types of sensor units based on a recognition result obtained by the recognition unit.

(8)

The medical observation system according to any one of (3) to (7),

wherein the judgment unit makes a judgment on the reliability of each of the plurality of types of sensor units based on map information regarding the internal environment before surgery.

(9)

The medical observation system according to (8),

in which map information regarding the internal environment before surgery is generated based on at least one of magnetic resonance imaging (MRI) or computed tomography (CT).

(10)

The medical observation system according to (8) or (9),

wherein the judgment unit makes a judgment on the reliability of each of the plurality of types of sensor units based on the map information regarding the internal environment before surgery and based on the environment map.

(11)

The medical observation system according to any one of (3) to (10),

wherein one of the plurality of types of sensor unit is an image sensor that images the internal environment,

the medical observation system further comprises a recognition unit that recognizes a status based on a video image acquired from the image sensor, and

the judgment unit makes a judgment on the reliability of each of the plurality of types of sensor units based on a priority set in advance for each of the plurality of types of sensor units, a recognition result obtained by the recognition unit, map information regarding the internal environment before surgery, and the environment map.

(12)

The medical observation system according to any one of (3) to (11), comprising a detection unit that detects a failure or deterioration in reliability of at least part of the sensor units based on a judgment result obtained by the judgment unit.

(13)

The medical observation system according to (12),

wherein the detection unit detects deterioration in the reliability of the entire medical observation system.

(14)

The medical observation system according to (13),

wherein, when the detection unit has detected the deterioration in the reliability of the entire medical observation system,

the generation unit generates an environment map based on a measurement result of the sensor unit having relatively high reliability among the plurality of types of sensor units.

(15)

The medical observation system according to (13) or (14), comprising a notification unit that notifies deterioration in reliability of the environment map when the detection unit has detected the deterioration in the reliability of the entire medical observation system.

(16)

The medical observation system according to (14) or (15), comprising an instruction unit that gives an instruction of operation of an autonomously driven main body in accordance with the environment map generated based on the measurement result obtained by the sensor unit having relatively high reliability among the plurality of types of sensor units, when the detection unit has detected deterioration in the reliability of the entire medical observation system.

(17)

The medical observation system according to (16),

wherein the instruction unit causes the main body to execute crisis avoidance operation.

(18)

The medical observation system according to (7),

wherein the plurality of types of sensor units are provided in an endoscope, and includes a stereo image sensor and a Time of Flight (ToF) sensor, and

the judgment unit judges deterioration in reliability of the ToF sensor due to mist based on a recognition result obtained by the recognition unit.

(19)

The medical observation system according to (18),

wherein the judgment unit detects contamination on a distal end portion of a lens barrel of the endoscope based on a value of the ToF sensor.

(20)

The medical observation system according to (18) or (19),

in which the judgment unit detects a light source defect in an imaging range of the stereo image sensor based on a result of comparison between the value of the stereo image sensor and the value of the ToF sensor.

(21)

The medical observation system according to any one of (1) to (20), comprising a support arm device having an arm unit at least a part of which is configured to be bendable and which is configured to be able to support a medical instrument,

wherein the plurality of types of sensor units are supported by the arm unit.

(22)

A control device comprising:

an acquisition unit that acquires individual sensor values of a plurality of types of sensor units that measure information regarding an internal environment;

a comparison unit that compares the individual sensor values of the plurality of types of sensor units acquired by the acquisition unit; and

a determination unit that determines a sensor unit to be used for observing the internal environment among the plurality of types of sensor units based on a comparison result obtained by the comparison unit.

(23)

A control method comprising:

acquiring individual sensor values of a plurality of types of sensor units that measure information regarding an internal environment;

comparing the acquired individual sensor values of the plurality of types of sensor units; and

determining a sensor unit to be used for observing the internal environment among the plurality of types of sensor units based on a comparison result.

### REFERENCE SIGNS LIST

1, 1A MEDICAL OBSERVATION SYSTEM
100 CONTROL DEVICE
110 COMMUNICATION UNIT
120 STORAGE UNIT
130 CONTROL UNIT
131 ACQUISITION UNIT
132 COMPARISON UNIT
133 JUDGMENT UNIT
134 DETERMINATION UNIT
135 GENERATION UNIT
136 RECOGNITION UNIT
137 DETECTION UNIT
138 NOTIFICATION UNIT
139 INSTRUCTION UNIT
140 COMMUNICATION CONTROL UNIT

1. A medical observation system comprising:
a plurality of types of sensor units that measure information regarding an internal environment;
an acquisition unit that acquires individual sensor values of the plurality of types of sensor units;
a comparison unit that compares the individual sensor values of the plurality of types of sensor units acquired by the acquisition unit; and
a determination unit that determines a sensor unit to be used for observing the internal environment among the plurality of types of sensor units based on a comparison result obtained by the comparison unit.

2. The medical observation system according to claim 1, comprising a generation unit that generates an environment map of the internal environment based on a measurement result obtained by the sensor unit determined by the determination unit.

3. The medical observation system according to claim 2, comprising a judgment unit that makes a judgment on reliability of each of the plurality of types of sensor units based on the comparison result obtained by the comparison unit,
wherein the determination unit determines the sensor unit to be used for observing the internal environment among the plurality of types of sensor units based on a judgment result obtained by the judgment unit.

4. The medical observation system according to claim 3,
wherein the determination unit determines at least two types of sensor units with high reliability as the sensor units to be used for observing the internal environment based on the judgment result obtained by the judgment unit.

5. The medical observation system according to claim 3,
wherein the judgment unit makes a judgment on the reliability of each of the plurality of types of sensor units based on priority set in advance for each of the plurality of types of sensor units.

6. The medical observation system according to claim 3,
wherein one of the plurality of types of sensor units is an image sensor that images the internal environment,
the medical observation system further comprises a recognition unit that recognizes a status based on a video image acquired from the image sensor, and
the judgment unit makes a judgment on the reliability of each of the plurality of types of sensor units based on a recognition result obtained by the recognition unit.

7. The medical observation system according to claim 3,
wherein the judgment unit makes a judgment on the reliability of each of the plurality of types of sensor units based on map information regarding the internal environment before surgery.

8. The medical observation system according to claim 7,
wherein the judgment unit makes a judgment on the reliability of each of the plurality of types of sensor units based on the map information regarding the internal environment before surgery and based on the environment map.

9. The medical observation system according to claim 3,
wherein one of the plurality of types of sensor unit is an image sensor that images the internal environment,
the medical observation system further comprises a recognition unit that recognizes a status based on a video image acquired from the image sensor, and
the judgment unit makes a judgment on the reliability of each of the plurality of types of sensor units based on a priority set in advance for each of the plurality of types of sensor units, a recognition result obtained by the recognition unit, map information regarding the internal environment before surgery, and the environment map.

10. The medical observation system according to claim 3, comprising a detection unit that detects a failure or deterioration in reliability of at least part of the sensor units based on a judgment result obtained by the judgment unit.

11. The medical observation system according to claim 10,
wherein the detection unit detects deterioration in the reliability of the entire medical observation system.

12. The medical observation system according to claim 11,
wherein, when the detection unit has detected the deterioration in the reliability of the entire medical observation system,
the generation unit generates an environment map based on a measurement result of the sensor unit having relatively high reliability among the plurality of types of sensor units.

13. The medical observation system according to claim 11, comprising a notification unit that notifies deterioration in reliability of the environment map when the detection unit has detected the deterioration in the reliability of the entire medical observation system.

14. The medical observation system according to claim 11, comprising an instruction unit that gives an instruction of operation of an autonomously driven main body in accordance with the environment map generated based on the measurement result obtained by the sensor unit having relatively high reliability among the plurality of types of sensor units, when the detection unit has detected deterioration in the reliability of the entire medical observation system.

15. The medical observation system according to claim 14,
wherein the instruction unit causes the main body to execute crisis avoidance operation.

16. The medical observation system according to claim 6,
wherein the plurality of types of sensor units are provided in an endoscope, and includes a stereo image sensor and a Time of Flight (ToF) sensor, and
the judgment unit judges deterioration in reliability of the ToF sensor due to mist based on a recognition result obtained by the recognition unit.

17. The medical observation system according to claim 16,
wherein the judgment unit detects contamination on a distal end portion of a lens barrel of the endoscope based on a value of the ToF sensor.

18. The medical observation system according to claim 1, comprising a support arm device having an arm unit at least a part of which is configured to be bendable and which is configured to be able to support a medical instrument, wherein the plurality of types of sensor units are supported by the arm unit.

19. A control device comprising:
an acquisition unit that acquires individual sensor values of a plurality of types of sensor units that measure information regarding an internal environment;
a comparison unit that compares the individual sensor values of the plurality of types of sensor units acquired by the acquisition unit; and
a determination unit that determines a sensor unit to be used for observing the internal environment among the plurality of types of sensor units based on a comparison result obtained by the comparison unit.

20. A control method comprising:
acquiring individual sensor values of a plurality of types of sensor units that measure information regarding an internal environment;
comparing the acquired individual sensor values of the plurality of types of sensor units; and
determining a sensor unit to be used for observing the internal environment among the plurality of types of sensor units based on a comparison result.
